SEQUENCE LISTING (52 sequences)

SEQ ID NO: 1 - DNA, 27 bp, Artificial Sequence
  RT primer of BRCA1
gtccaaagcgagcaagagaatcccagg

SEQ ID NO: 2 - DNA, 22 bp, Artificial Sequence
  RT primer of DPYD
gccactgaagagggacccatta

SEQ ID NO: 3 - DNA, 27 bp, Artificial Sequence
  RT primer of EGFR
cctggactatgtccgggaacacaaaga

SEQ ID NO: 4 - DNA, 23 bp, Artificial Sequence
  RT primer of PTEN
cagatggaaggggtggaactgtg

SEQ ID NO: 5 - DNA, 22 bp, Artificial Sequence
  RT primer of ERCC1
aaggcctatgagcagaaaccag

SEQ ID NO: 6 - DNA, 23 bp, Artificial Sequence
  RT primer of STMN1
agctgcagaagaaagacgcaagt

SEQ ID NO: 7 - DNA, 25 bp, Artificial Sequence
  RT primer of TUBB3
ttgccgccctcctgcagtatttatg

SEQ ID NO: 8 - DNA, 20 bp, Artificial Sequence
  RT primer of RRM1
gctactggcagctacattgc

SEQ ID NO: 9 - DNA, 24 bp, Artificial Sequence
  RT primer of VEGFR
ggacttcctgaccttggagcatct

SEQ ID NO: 10 - DNA, 23 bp, Artificial Sequence
  RT primer of HER2
catttctgccggagagctttgat

SEQ ID NO: 11 - DNA, 23 bp, Artificial Sequence
  RT primer of TYMP
aatgtcatccagagcccagagca

SEQ ID NO: 12 - DNA, 28 bp, Artificial Sequence
  RT primer of TYMS
aacctaacgtgtgttctggaagggtgtt

SEQ ID NO: 13 - DNA, 25 bp, Artificial Sequence
  RT primer of PDGFR
cggacaagtgacccaagtgctgaag

SEQ ID NO: 14 - DNA, 20 bp, Artificial Sequence
  RT primer of TOP2A
tcgtcagaacatggacccag

SEQ ID NO: 15 - DNA, 25 bp, Artificial Sequence
  RT primer of GUSB
catgccagttccctccagcttcaat

SEQ ID NO: 16 - DNA, 20 bp, Artificial Sequence
  RT primer of TBP
tttaacttcgcttccgctgg

SEQ ID NO: 17 - DNA, 29 bp, Artificial Sequence
  RT primer of PSMC4
tcagaccagaagccagatgtgatgtacgc

SEQ ID NO: 18 - DNA, 27 bp, Artificial Sequence
  RT primer of B2M
aaagatgagtatgcctgccgtgtgaac

SEQ ID NO: 19 - DNA, 29 bp, Artificial Sequence
  RT primer of b-actin
ggggcgccccacgatggaggggaagacgg

SEQ ID NO: 20 - DNA, 29 bp, Artificial Sequence
  RT primer of GAPDH
ttaccagagttaaaagcagccctggtgac

SEQ ID NO: 21 - DNA, 30 bp, Artificial Sequence
  RT primer of RPL37A
ttcaactccttcagtcttctgatggcggac

SEQ ID NO: 22 - DNA, 21 bp, Artificial Sequence
  RT primer of Artificial RNA
tcatcctgatcgacaagaccg

SEQ ID NO: 23 - DNA, 27 bp, Artificial Sequence
  PCR primer of BRCA1
ccatccattccagttgatctgtgggca

SEQ ID NO: 24 - DNA, 24 bp, Artificial Sequence
  PCR primer of DPYD
gaaagccaggatcaaagtctcagt

SEQ ID NO: 25 - DNA, 26 bp, Artificial Sequence
  PCR primer of EGFR
cgacggtcctccaagtagttcatgcc

SEQ ID NO: 26 - DNA, 23 bp, Artificial Sequence
  PCR primer of PTEN
aactggcaggtagaaggcaactc

SEQ ID NO: 27 - DNA, 21 bp, Artificial Sequence
  PCR primer of ERCC1
ttccagagaccgggagacgaa

SEQ ID NO: 28 - DNA, 23 bp, Artificial Sequence
  PCR primer of STMN1
agcacttctttctcgtgctctcg

SEQ ID NO: 29 - DNA, 25 bp, Artificial Sequence
  PCR primer of TUBB3
gtcaggcctggagctgcaataagac

SEQ ID NO: 30 - DNA, 20 bp, Artificial Sequence
  PCR primer of RRM1
tctcagcatcggtacaaggc

SEQ ID NO: 31 - DNA, 24 bp, Artificial Sequence
  PCR primer of VEGFR
ctgtggatacactttcgcgatgcc

SEQ ID NO: 32 - DNA, 22 bp, Artificial Sequence
  PCR primer of HER2
caaacacttggagctgctctgg

SEQ ID NO: 33 - DNA, 23 bp, Artificial Sequence
  PCR primer of TYMP
acgaaccagctgctcactctgac

SEQ ID NO: 34 - DNA, 28 bp, Artificial Sequence
  PCR primer of TYMS
ttggcatcccagattttcactcccttgg

SEQ ID NO: 35 - DNA, 26 bp, Artificial Sequence
  PCR primer of PDGFR
gaagttccctacgaagccctgtttgc

SEQ ID NO: 36 - DNA, 21 bp, Artificial Sequence
  PCR primer of TOP2A
tcttctcggtgccattcaaca

SEQ ID NO: 37 - DNA, 24 bp, Artificial Sequence
  PCR primer of GUSB
aggatcacctcccgttcgtaccac

SEQ ID NO: 38 - DNA, 21 bp, Artificial Sequence
  PCR primer of TBP
cgccaagaaacagtgatgctg

SEQ ID NO: 39 - DNA, 28 bp, Artificial Sequence
  PCR primer of PSMC4
ctgcttgtagagctcgaaatgcgtgagc

SEQ ID NO: 40 - DNA, 27 bp, Artificial Sequence
  PCR primer of B2M
ggcatcttcaaacctccatgatgctgc

SEQ ID NO: 41 - DNA, 20 bp, Artificial Sequence
  Forward PCR primer of pcDNA
tacatcaatgggcgtggata

SEQ ID NO: 42 - DNA, 20 bp, Artificial Sequence
  Reverse PCR primer of pcDNA
ggcggagttgttacgacatt

SEQ ID NO: 43 - DNA, 29 bp, Artificial Sequence
  PCR primer of b-actin
tcaccatggatgatgatatcgccgcgctc

SEQ ID NO: 44 - DNA, 30 bp, Artificial Sequence
  PCR primer of GAPDH
ggcgaggaagtcaggtggagcgaggctagc

SEQ ID NO: 45 - DNA, 30 bp, Artificial Sequence
  PCR primer of RPL37A
agatgaagagacgagctgtggggatctggc

SEQ ID NO: 46 - DNA, 21 bp, Artificial Sequence
  PCR primer of Artificial RNA
cttgctcctgccgagaaagta

SEQ ID NO: 47 - DNA, 18 bp, Artificial Sequence
  Forward universal primer
aggtgacactatagaata

SEQ ID NO: 48 - DNA, 19 bp, Artificial Sequence
  Reverse universal primer
gtacgactcactataggga

SEQ ID NO: 49 - DNA, 21 bp, Artificial Sequence
  Primer
ggccagcattaccatcagtgg

SEQ ID NO: 50 - DNA, 21 bp, Artificial Sequence
  Primer
ccactgaagagggacccatta

SEQ ID NO: 51 - DNA, 20 bp, Artificial Sequence
  Primer
gtttggcactgctcctgctg

SEQ ID NO: 52 - DNA, 21 bp, Artificial Sequence
  Primer
agccaggatcaaagtctcagt

(19) United States
(12) Patent Application Publication
(10) Pub. No.: US 2017/0051332 A1
(43) Pub. Date: Feb. 23, 2017

(54) METHODS AND COMPOSITIONS FOR DETECTING EXPRESSION OF TARGET GENES

(71) Applicant: NINGBO HEALTH GENE TECHNOLOGIES CO., LTD., Ningbo, Zhejiang (CN)

(72) Inventors: Yong WU, Ningbo City, Zhejiang (CN); Bing XU, Ningbo City, Zhejiang (CN); Linan WU, Ningbo City, Zhejiang (CN)

(73) Assignee: NINGBO HEALTH GENE TECHNOLOGIES CO., LTD., Ningbo City, Zhejiang (CN)

(21) Appl. No.: 14/376,407

(22) PCT Filed: May 4, 2014
(86) PCT No.: PCT/CN2014/076724

§ 371 Date: Aug. 1, 2014

(51) Int. Cl. C12Q 1/68

(52) U.S. Cl. CPC C12Q 1/686; C12Q 1/6886; C12Q 2600/158; C12Q 2600/16

(57) ABSTRACT

The present invention relates to methods and compositions, and uses thereof, for simultaneously detecting expression of multiple target genes in a sample. The present invention further relates to certain isolated polynucleotides that can be used as primers or primer pairs in the present methods and compositions for simultaneously detecting expression of multiple target genes in a sample.

### I. CROSS-REFERENCE TO RELATED APPLICATION

In certain aspect, this application relates to Chinese patent application No. 201310032768.2, filed Jan. 25, 2013, the content of which is incorporated by reference in its entirety.

### II. SUBMISSION OF SEQUENCE LISTING ON ASCII TEXT FILE

The content of the following submission on ASCII text file is incorporated herein by reference in its entirety: a computer readable form (CRF) of the Sequence Listing (file name: 737222000300SeqList.txt, date recorded: Aug. 1, 2014, size: 9,705 KB).

### III. TECHNICAL FIELD

The present invention relates to methods and compositions, and uses thereof, for simultaneously detecting expression of multiple target genes in a sample. The present invention further relates to certain isolated polynucleotides that can be used as primers or primer pairs in the present methods and compositions for simultaneously detecting expression of multiple target genes in a sample.

### IV. BACKGROUND ART

In recent years, pharmacogenetics/pharmacogenomics have made great progress in research on functional mechanisms of anti-tumor drugs. Scientists discovered that there was great relationship between tumor cell killing effect of some anti-tumor drugs and a certain kind (or group) of genes expression and/or polymorphism. It has been a reasonable choice to improve the curative effect and reduce invalid treatment by detecting related genes, predicating the effect of chemotherapy and targeting drugs, and choosing suitable drugs to personalize chemotherapy.

A large number of clinical data indicated that the expression levels of 14 genes including ERCC1, RRM1, TYMS, TUBB3 etc. in tumor tissue could predicate patients' responses to commonly used chemotherapeutic drugs such as Platinum, Gemcitabine, 5-Fu, and other anti-microtubule drugs. To formulate a personalized treatment plan according to the gene expression levels in tumor tissue of patient is conducive to improve targeted therapy by using patient-specific chemotherapy drugs. Choosing chemotherapy drugs by target detection can avoid ineffective or harmful chemotherapy, save treatment time and cost, improve life quality of patients. So it is necessary to develop a multiple gene expression detection technique to detect the 14 gene expression levels so that doctors can quickly provide important information to patients for much safer and more efficient treatment.

Currently, there are several traditional methods to detect gene expression (mRNA level).

### (1) PCR

Commonly used PCR detection methods are fluorescence qPCR, immunity PCR and RT-PCR. Fluorescence qPCR detection method is the most mature. The advantages of qPCR are high sensitivity as well as quantitative, however, the disadvantages are low throughput and high cost.

### (2) Molecular Hybridization—Northern Blotting and RNA In Situ Hybridization

Hybridization is the process of establishing a non-covalent, sequence-specific interaction between two or more complementary strands of nucleic acids into a single complex, which in the case of two strands is referred to as a duplex. Oligonucleotides, DNA, or RNA will bind to their complement under normal conditions. Hybridization is high sensitive and specific, so target sequence probes are used for the specific detection of known sequence. It is widely used in screening cloned gene, making restriction map, qualitative, quantitative detection of gene sequence and disease diagnosis. The disadvantages of molecular hybridization are complicated operation and low throughput.

### (3) Gene Chip (DNA Microarray)

Gene chip is a collection of microscopic DNA spots attached to a solid surface. Scientists use DNA microarrays to measure the expression levels of large numbers of genes simultaneously or to genotype multiple regions of a genome. Since an array can contain tens of thousands of probes, a microarray experiment can accomplish many genetic tests in parallel, however, high cost and the complexity of attaching probes limited its large-scale promotion. On the other hand, gene chip cannot be used to accurately quantify gene expression; scientists need large amount of nucleic acid in microarrays because of its low sensitivity.

### (4) Transcriptome Sequencing

Transcriptome sequencing refers to the use of high-throughput sequencing technologies to sequence cDNA in order to get information about a sample's RNA content. The technique has been adopted in studies of diseases like cancer. With deep coverage and base-level resolution, next-generation sequencing provides information on differential expression of genes, including gene alleles and differently spliced transcripts; non-coding RNAs; post-transcriptional mutations or editing; and gene fusions. Compared with the traditional DNA microarrays, transcriptome sequencing generates much more amount of digitized signal (data), but does not need to design probe oligos. However, the cost is 10-100 times high than microarrays, which might be the limitation for its application in clinical care.

In brief, the four techniques mentioned above cannot meet the demand for rapid, accurate detection of multiple gene expression. The present invention addresses this and other related needs in the field.

### V. DISCLOSURE OF THE INVENTION

In one aspect, the present disclosure provides for a method for simultaneously detecting expression of multiple target genes in a sample, which method comprises: a) obtaining total RNA or mRNA from a sample, said total RNA or mRNA comprising target RNA encoded by multiple target genes in said sample; b) obtaining a target cDNA corresponding to each of said target RNA from said total RNA or mRNA via reverse transcription using said total RNA or mRNA obtained in step a) as a template and a reverse transcription primer for each of said target RNA; c) obtaining an amplicon from each of said cDNA obtained in step b) via multiplex PCR using said cDNA as a template and a pair of PCR primers for amplifying each of said cDNA; and d) analyzing said multiple amplicons using capillary electrophoresis, wherein the sizes of said multiple amplicons range from about 50 base pairs (bp) to about 300 bp, the difference of said sizes between at least two adjacent amplicons in sizes is 2 or more bp, and said 2 or more bp size difference is generated using at least one spacer nucleotide in said reverse transcription primer and/or PCR primer(s), said spacer nucleotide(s) may or may not be complementary to the nucleotide(s) at the corresponding positions(s) of the target RNA and/or target cDNA, provided that when at least one of said spacer nucleotide(s) is complementary to the nucleotide(s) at the corresponding positions(s) of the target RNA and/or target cDNA, the sizes of said multiple amplicons range from about 50 base pairs (bp) to about 150 bp.

In another aspect, the present disclosure provides for a kit or system for simultaneously detecting expression of multiple target genes encoding multiple target RNA in a sample, which kit or system comprises: a) a reverse transcription primer for each of target RNA for obtaining a target cDNA corresponding to each of said target RNA via reverse transcription; b) a pair of PCR primers for amplifying each of said target cDNA to obtain an amplicon from each of said target cDNA via multiplex PCR; and d) means for analyzing said multiple amplicons using capillary electrophoresis, wherein the sizes of said multiple amplicons range from about 50 base pairs (bp) to about 300 bp, the difference of said sizes between at least two adjacent amplicons in sizes is 2 or more bp, and said 2 or more bp size difference is generated using at least one spacer nucleotide in said reverse transcription primer and/or PCR primer(s), said spacer nucleotide(s) may or may not be complementary to the nucleotide(s) at the corresponding positions(s) of the target RNA and/or target cDNA, provided that when at least one of said spacer nucleotide(s) is complementary to the nucleotide(s) at the corresponding positions(s) of the target RNA and/or target cDNA, the sizes of said multiple amplicons range from about 50 base pairs (bp) to about 150 bp.

In some embodiments, the present disclosure relates to methods and compositions for simultaneously or synchronously detecting multiple gene expression levels, and the uses of the methods and compositions on formalin-fixed, paraffin-embedded (FFPE) samples, e.g., a 14 gene expression detection kit for anticancer drugs medication guide and its detection method.

In still another aspect, the present disclosure provides for an isolated polynucleotide which comprises a polynucleotide sequence that exhibits at least 70%, 75%, 80%, 85%, 90%, 95%, 99% or 100% identity to any of the RRM1, TOP2A, DPYD, ERCC1, HER2, PTEN, STMN1, TYMP, VEGFR, TUBB3, PDGFRA, EGFR, BRCA1, TYMS, TBP, GUSB, B2M and PSMC4 polynucleotide sequences set forth in Table 7, wherein said polynucleotide does not comprise a wild-type, full length RRM1, TOP2A, DPYD, ERCC1, HER2, PTEN, STMN1, TYMP, VEGFR, TUBB3, PDGFRA, EGFR, BRCA1, TYMS, TBP, GUSB, B2M and PSMC4 polynucleotide sequence from which said polynucleotide is derived.

In yet another aspect, the present disclosure provides for a primer composition, which primer composition comprises, consists essentially of or consists of any of the primer pairs set forth in Table 7.

### VI. BRIEF DESCRIPTION OF THE DRAWINGS

FIG. 1 illustrates an exemplary, schematic diagram of a standard curve.

FIG. 2 illustrates an exemplary electropherogram of detection result of a patient sample. Nineteen (19) peaks corresponding to 14 genes that are related with anticancer drug medication guide, 4 RNA reference gene peaks and a PCR control gene (pcDNA) peak, are shown. Please refer to Table 9 for the detected gene names and corresponding fragment size.

FIG. 3 illustrates an exemplary electropherogram of detection result of a patient sample using the 14 gene expression detection kit with 7 internal RNA control genes. Twenty-two (22) peaks corresponding to 14 genes that are related with anticancer drug medication guide, 7 RNA reference gene peaks and a RNA reaction control peak, are shown. Please refer to Table 7 for the detected gene names and corresponding fragment size.

### VII. MODES OF CARRYING OUT THE INVENTION

### A. Definitions

Unless defined otherwise, all technical and scientific terms used herein have the same meaning as is commonly understood by one of ordinary skill in the art to which this invention belongs. All patents, patent applications (published or unpublished), and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “mammal” refers to any of the mammalian class of species. Frequently, the term “mammal,” as used herein, refers to humans, human subjects or human patients.

As used herein, the term “subject” is not limited to a specific species or sample type. For example, the term “subject” may refer to a patient, and frequently a human patient. However, this term is not limited to humans and thus encompasses a variety of mammalian species.

As used herein the term “sample” refers to anything which may contain an analyte for which an analyte assay is desired. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

The terms “polynucleotide,” “oligonucleotide,” “nucleic acid” and “nucleic acid molecule” are used interchangeably herein to refer to a polymeric form of nucleotides of any length, e.g., at least 8, 9, 10, 20, 30, 40, 50, 100, 200, 300, 400, 500, 1,000 or more nucleotides, and may comprise ribonucleotides, deoxyribonucleotides, analogs thereof, or mixtures thereof. This term refers only to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), including tRNA, rRNA, hRNA, and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids (“PNAs”)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. Thus, these terms include, for example, 3′-deoxy-2′,5′-DNA, oligodeoxyribonucleotide N3′ to P5′ phosphoramidates, 2′-O-alkyl-substituted RNA, hybrids between DNA and RNA or between PNAs and DNA or RNA, and also include known types of modifications, for example, labels, alkylation, “caps,” substitution of one or more of the nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters), those containing pendant moieties, such as, for example, proteins (including enzymes (e.g. nucleases), toxins, antibodies, signal peptides, poly-L-lysine, etc.), those with intercalators (e.g., acridine, psoralen, etc.), those containing chelates (of, e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

It will be appreciated that, as used herein, the terms “nucleoside” and “nucleotide” will include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides can also include modifications on the sugar moiety, e.g., wherein one or more of the hydroxyl groups are replaced with halogen, aliphatic groups, or are functionalized as ethers, amines, or the like. The term “nucleotidic unit” is intended to encompass nucleosides and nucleotides.

“Nucleic acid probe” and “probe” are used interchangeably and refer to a structure comprising a polynucleotide, as defined above, that contains a nucleic acid sequence that can bind to a corresponding target. The polynucleotide regions of probes may be composed of DNA, and/or RNA, and/or synthetic nucleotide analogs.

As used herein, “complementary or matched” means that two nucleic acid sequences have at least 50% sequence identity. Preferably, the two nucleic acid sequences have at least 60%, 70%, 80%, 90%, 95%, 96%, 97%, 98%, 99% or 100% of sequence identity. “Complementary or matched” also means that two nucleic acid sequences can hybridize under low, middle and/or high stringency condition(s).

As used herein, “substantially complementary or substantially matched” means that two nucleic acid sequences have at least 90% sequence identity. Preferably, the two nucleic acid sequences have at least 95%, 96%, 97%, 98%, 99% or 100% of sequence identity. Alternatively, “substantially complementary or substantially matched” means that two nucleic acid sequences can hybridize under high stringency condition(s).

In general, the stability of a hybrid is a function of the ion concentration and temperature. Typically, a hybridization reaction is performed under conditions of lower stringency, followed by washes of varying, but higher, stringency. Moderately stringent hybridization refers to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions are conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C. Low stringency hybridization refers to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M EDTA. Other suitable moderate stringency and high stringency hybridization buffers and conditions are well known to those of skill in the art and are described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999).

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See Kanehisa (1984) Nucleic Acids Res. 12:203-215.

As used herein, “biological sample” refers to any sample obtained from a living or viral source or other source of macromolecules and biomolecules, and includes any cell type or tissue of a subject from which nucleic acid or protein or other macromolecule can be obtained. The biological sample can be a sample obtained directly from a biological source or a sample that is processed. For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants and processed samples derived therefrom. Also included are soil and water samples and other environmental samples, viruses, bacteria, fungi, algae, protozoa and components thereof.

It is understood that aspects and embodiments of the invention described herein include “consisting” and/or “consisting essentially of” aspects and embodiments.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Other objects, advantages and features of the present invention will become apparent from the following specification taken in conjunction with the accompanying drawings.

### B. Methods for Simultaneously Detecting Expression of Target Genes in a Sample

In one aspect, the present disclosure provides for a method for simultaneously detecting expression of multiple target genes in a sample, which method comprises: a) obtaining total RNA or mRNA from a sample, said total RNA or mRNA comprising target RNA encoded by multiple target genes in said sample; b) obtaining a target cDNA corresponding to each of said target RNA from said total RNA or mRNA via reverse transcription using said total RNA or mRNA obtained in step a) as a template and a reverse transcription primer for each of said target RNA; c) obtaining an amplicon from each of said cDNA obtained in step b) via multiplex PCR using said cDNA as a template and a pair of PCR primers for amplifying each of said cDNA; and d) analyzing said multiple amplicons using capillary electrophoresis, wherein the sizes of said multiple amplicons range from about 50 base pairs (bp) to about 300 bp, the difference of said sizes between at least two adjacent amplicons in sizes is 2 or more bp, and said 2 or more bp size difference is generated using at least one spacer nucleotide in said reverse transcription primer and/or PCR primer(s), said spacer nucleotide(s) may or may not be complementary to the nucleotide(s) at the corresponding positions(s) of the target RNA and/or target cDNA, provided that when at least one of said spacer nucleotide(s) is complementary to the nucleotide(s) at the corresponding positions(s) of the target RNA and/or target cDNA, the sizes of said multiple amplicons range from about 50 base pairs (bp) to about 150 bp.

The reverse transcription primer and the PCR primers can be designed by any suitable methods in the art. Often, factors such as amplicon length, product position, melting temperature (Tₘ) of the product, optimum annealing temperature (Tₐ Opt), and primer pair Tm mismatch calculation, can be considered in designing the primers. In some embodiments, the primers can be designed using known primer design software, e.g., Primer Premier, AlleleID®, and Beacon Designer™. Often, spacer nucleotide(s) can be added to the reverse transcription primer and/or the PCR primers to control or increase amplicon length. In some embodiments, spacer nucleotide(s) are not added to the reverse transcription primer and/or the PCR primers for any factors that are not related to amplicon length. In some embodiments, spacer nucleotide(s) are not added to the reverse transcription primer and/or the PCR primers but for the goal to control or increase amplicon length so that the lengths of various amplicons can be sufficiently distinguished in capillary electrophoresis.

The spacer nucleotide(s) can be distributed between or among the reverse transcription primer and the PCR primer(s) in any suitable manner. In some embodiments, the reverse transcription primer comprises at least one spacer nucleotide and the pair of PCR primers does not comprise any spacer nucleotide. In other embodiments, the reverse transcription primer does not comprise any spacer nucleotide and at least one of the pair of PCR primers comprises at least one spacer nucleotide. In still other embodiments, both of the pair of PCR primers comprise at least one spacer nucleotide. The spacer nucleotide(s) can be distributed between or among the PCR primers in any suitable manner. For example, the total number of the spacer nucleotides can be distributed between the two PCR primers to minimize the size difference of the two PCR primers.

In some embodiments, the reverse transcription primer comprises at least one spacer nucleotide and at least one of the pair of PCR primers comprises at least one spacer nucleotide. The spacer nucleotide(s) can be distributed between or among the reverse transcription primer and the PCR primer(s) in any suitable manner. For example, the total number of the spacer nucleotides can be distributed between the reverse transcription primer and the at least one of the pair of PCR primers to minimize the size difference of the reverse transcription primer and the at least one of the pair of PCR primers. In other embodiments, both of the pair of PCR primers comprise at least one spacer nucleotide. The spacer nucleotide(s) can be distributed between or among the PCR primers in any suitable manner. For example, the total number of the spacer nucleotides can be distributed between the two PCR primers to minimize the size difference of the two PCR primers.

The reverse transcription primer can comprise any suitable number of spacer nucleotide(s). For example, the reverse transcription primer can comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 spacer nucleotide(s). The spacer nucleotide(s) can be located at any suitable position of the reverse transcription primer. For example, the spacer nucleotide(s) can be located at the 5′ end of the reverse transcription primer.

The reverse transcription primer can comprise any suitable number of nucleotides. In some embodiments, the reverse transcription primer can comprise at least 16 nucleotides. In other embodiments, the gene specific portion of the reverse transcription primer can comprise at least 16 nucleotides. In still other embodiments, the reverse transcription primer can comprise 16 to 50 nucleotides. In yet other embodiments, the spacer nucleotide(s) is located at the 5′ end of the reverse transcription primer that comprises at least 16 nucleotides, e.g., at least 17, 18, 19, 20, 25, 30 or more nucleotides, in the non-spacer portion.

The PCR primer(s) in the pair of PCR primers can comprise any suitable number of spacer nucleotide(s). For example, one or both members of the pair of PCR primers can comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 spacer nucleotide(s). The spacer nucleotide(s) can be located at any suitable position of the PCR primer(s). For example, the spacer nucleotide(s) can be located at the 5′ end of one or both members of the pair of PCR primers.

The PCR primer(s) can comprise any suitable number of nucleotides. In some embodiments, one or both members of the pair of PCR primers can comprise at least 16 nucleotides. In other embodiments, the gene specific portion of one or both members of the pair of PCR primers can comprise at least 16 nucleotides, e.g., at least 17, 18, 19, 20, 25, 30 or more nucleotides. In still other embodiments, one or both members of the pair of PCR primers can comprise 16 to 50 nucleotides. In yet other embodiments, the spacer nucleotide(s) can be located at the 5′ end of one or both members of the pair of PCR primers that comprise at least 16 nucleotides, e.g., at least 17, 18, 19, 20, 25, 30 or more nucleotides, in the non-spacer portion.

The sizes of the multiple amplicons can have any suitable range. In some embodiments, the sizes of the multiple amplicons can range from about 50 bp to about 150 bp, e.g., about 50-140, 50-130, 50-120, 50-110 or 50-100 bp. In other embodiments, the sizes of the multiple amplicons range from about 60 bp to about 120 bp, e.g., about 60-110, 60-100, 70-120, 70-110 or 70-100 bp.

The difference of the sizes between at least two adjacent amplicons in sizes can have any suitable range. In some embodiments, the difference of the sizes between at least two adjacent amplicons in sizes can be 3 or more bp, e.g., 3, 4, 5, 6, 7, 8, 9 or 10 bp. In other embodiments, the difference of the sizes between at least a quarter of any adjacent amplicons in sizes can be 3 or more bp, e.g., 3, 4, 5, 6, 7, 8, 9 or 10 bp. In still other embodiments, the difference of the sizes between at least half of any adjacent amplicons can be 3 or more bp, e.g., 3, 4, 5, 6, 7, 8, 9 or 10 bp. In yet other embodiments, the difference of the sizes between all of any adjacent amplicons in sizes can be 3 or more bp, e.g., 3, 4, 5, 6, 7, 8, 9 or 10 bp.

In some embodiments, the reverse transcription primer is used in the reverse transcription step to obtain cDNA only, but is not used in the subsequent multiplex PCR step to obtain the amplicons. In other embodiments, the reverse transcription primer is used in the reverse transcription step to obtain cDNA, and is also used as one of the PCR primers in the subsequent multiplex PCR step to obtain the amplicons.

In some embodiments, the reverse transcription primer can further comprise a first tag sequence. In other embodiments, one or both members of the pair of PCR primers can comprise a sequence that is identical to the sequence of the first tag sequence. In still other embodiments, the other member of the pair of PCR primers can comprise a second tag sequence.

In some embodiments, one member of the pair of PCR primers comprises the reverse transcription primer that further comprises a first tag sequence and the other member of the pair of PCR primers comprises a sequence that is substantially complementary to a portion of the target cDNA and a second tag sequence. Using the above pair of PCR primers, the first PCR cycle or first two PCR cycles can generate a double-stranded DNA that comprises a sequence that is substantially complementary to the first tag sequence and the second tag sequence at its 5′ end and 3′ end, respectively. In other embodiments, the third and subsequent PCR cycles can use a pair of PCR primers that comprises one PCR primer that comprises a sequence that is identical to the sequence of the first tag sequence and another PCR primer comprises a sequence that is identical to the sequence of the second tag sequence.

In some embodiments, amplification of at least a quarter, half or all of the cDNA involves the use of two pairs of PCR primers: a) one member of the first pair of PCR primers comprising the reverse transcription primer that further comprises a first tag sequence, and the other member of the first pair of PCR primers comprising a sequence that is substantially complementary to a portion of the target cDNA and a second tag sequence; and b) one member of the second pair of PCR primers comprising a sequence that is identical to the sequence of the first tag sequence, and the other member of the second pair of PCR primers comprising a sequence that is identical to the sequence of the second tag sequence.

In some embodiments, the present methods can further comprise assessing expression of an internal reference gene. The present methods can further comprise assessing expression of any suitable number of internal reference genes, e.g., at least 2, 3, 4, 5, 6, 7, 8, 9, or 10 internal reference genes.

In some embodiments, the present methods can further comprise analyzing an amplicon of a PCR control polynucleotide. Any suitable PCR control polynucleotide can be used, e.g., a plasmid DNA such as pcDNA.

In some embodiments, relative expression levels of multiple target genes and/or the internal reference gene(s) can be assessed based on a standard curve of each of the target genes and/or the internal reference gene(s). The standard curve can be established by any suitable methods. For example, the standard curve can be established based on a plot between a peak ratio (“R”) of each of the target genes and/or the internal reference gene(s) over the amplicon of PCR control polynucleotide, and a function of R (f(R)). The data points in the standard curve can be obtained or selected by any suitable methods. For example, the data points in the standard curve can be obtained or selected by curve fitting of multiple data points.

In some embodiments, the relative expression levels “κ” of a target gene can be calculated using the following formulae: κ=n*f(R_{g})/Σᵢ₌₀ⁿfᵢ(Rᵢ) (n=4), where R_{g} is peak ratio of a target gene and the amplicon of PCR control polynucleotide (e.g., pcDNA), R_{g}=A_{g}/A_{pcDNA}, Rᵢ is peak ratio of an internal reference gene and the amplicon of PCR control polynucleotide (e.g., pcDNA), Rᵢ=Aᵢ/A_{pcDNA}.

The present methods can be used for simultaneously detecting expression of any suitable number of target genes in a sample. For example, the present methods can be used for simultaneously detecting expression of at least 2, 3, 4, 5, 6, 7, 8, 9, 10, 11, 12, 13, 14, 15, 16, 17, 18, 19, 20, 21, 22, 23, 24, 25, 26, 27, 28, 29 or 30 target genes in a sample.

The present methods can be used for any suitable purpose. For example, the present methods can be used for simultaneously detecting expression of the target genes that is associated with a therapy, e.g., a tumor or cancer therapy. In some embodiments, the present methods can be used for simultaneously detecting expression of the target genes that relates to the toxicity, ADR, efficacy and/or dosage of an anti-tumor or anti-cancer drug. In other embodiments, the present methods can be used for simultaneously detecting expression of the target genes that relates to the toxicity, ADR, efficacy and/or dosage of multiple anti-tumor or anti-cancer drugs.

The present methods can be used for simultaneously detecting expression of any suitable target genes. Exemplary target genes include ribonucleotide reductase M1 (RRM1), topoisomerase (DNA) II alpha (TOP2A), dihydropyrimidine dehydrogenase (DPYD), excision repair cross-complementing rodent repair deficiency, complementation group 1 (ERCC1), v-erb-b2 erythroblastic leukemia viral oncogene homolog 2 (HER2), phosphatase and tensin homolog (PTEN), stathmin 1 (STMN1), thymidine phosphorylase (TYMP), kinase insert domain receptor (VEGFR), tubulin beta 3 class III (TUBB3), platelet-derived growth factor receptor, alpha polypeptide (PDGFRA), epidermal growth factor receptor (EGFR), breast cancer 1 (BRCA1), and thymidylate synthetase (TYMS). The present methods can be used for simultaneously detecting expression of 2, 3, 4, 5, 6, 7, 8, 9, 10, 11, 12, 13 or 14 of the above target genes.

In some embodiments, the present methods can comprise simultaneously detecting expression of any suitable internal reference gene(s). Exemplary internal reference genes include TATA-binding protein (TBP), beta-glucuronidase (GUSB), β₂ microglobulin (B2M) and 26S protease regulatory subunit 6B (PSMC4). The present methods can be used for simultaneously detecting expression of 1, 2, 3 or 4 of the above internal reference genes.

The present methods can be used for simultaneously detecting expression of target genes in any suitable sample. For example, the sample can be a biological sample. In some embodiments, the biological sample can be obtained or derived from a human or a non-human mammal. The present methods can be used for simultaneously detecting expression of any suitable target genes in any suitable biological sample, e.g., a whole blood, a plasma, a fresh blood, a blood not containing an anti-coagulate, a urine, a saliva sample, mucosal cells, and cells from a human or a non-human mammal. In some embodiments, the sample is a formalin-fixed, paraffin-embedded (FFPE) sample.

The reverse transcription primer and/or PCR primer(s) can comprise any suitable number of spacer nucleotide(s) that is not complementary to the nucleotide(s) at the corresponding positions(s) of the target RNA and/or target cDNA. In some embodiments, at least one spacer nucleotide in the reverse transcription primer and/or PCR primer(s) is not complementary to the nucleotide(s) at the corresponding positions(s) of the target RNA and/or target cDNA. In other embodiments, at least 2, 3, 4, 5, 6, 7, 8, 9, 10, 11, 12, 13, 14, 15, 16, 17, 18, 19, 20, 21, 22, 23, 24, 25, 26, 27, 28, 29 or 30 spacer nucleotides in the reverse transcription primer and/or PCR primer(s) are not complementary to the nucleotide(s) at the corresponding positions(s) of the target RNA and/or target cDNA.

Any suitable number of the reverse transcription primers and/or PCR primers can comprise at least one spacer nucleotide that is not complementary to the nucleotide(s) at the corresponding positions(s) of the target RNA and/or target cDNA. In some embodiments, at least a quarter, half or all of the reverse transcription primers and/or PCR primers comprise at least one spacer nucleotide that is not complementary to the nucleotide(s) at the corresponding positions(s) of the target RNA and/or target cDNA. In other embodiments, at least a quarter, half or all of the reverse transcription primers and/or PCR primers comprise at least 2, 3, 4, 5, 6, 7, 8, 9, 10, 11, 12, 13, 14, 15, 16, 17, 18, 19, 20, 21, 22, 23, 24, 25, 26, 27, 28, 29 or 30 spacer nucleotides that are not complementary to the nucleotides at the corresponding positions(s) of the target RNA and/or target cDNA.

In some embodiments, the present methods can further comprise obtaining an internal reaction control cDNA via reverse transcription using an internal reaction control RNA as a template and a reverse transcription primer for the internal reaction control RNA, obtaining an amplicon from the internal reaction control cDNA via PCR using the internal reaction control cDNA as a template and a pair of PCR primers for amplifying the internal reaction control cDNA, and analyzing the amplicon from the internal reaction control cDNA using capillary electrophoresis. Any suitable internal reaction control RNA can be used in the present methods. For example, the internal reaction control RNA can be an artificial RNA, e.g., an antisense RNA of kanamycin resistance gene.

The present methods can be used for simultaneously detecting expression of any suitable target genes. Exemplary target genes include the target genes RRM1, TOP2A, Beta-type platelet-derived growth factor receptor (PDGFRB), ERCC1, HER2, PTEN, STMN1, excision repair cross-complementing rodent repair deficiency, complementation group 2 (ERCC2 or XPD), VEGFR, TUBB3, DPYD, EGFR, BRCA1, and TYMS. The present methods can be used for simultaneously detecting expression of 2, 3, 4, 5, 6, 7, 8, 9, 10, 11, 12, 13 or 14 of the above target genes.

In some embodiments, the present methods can comprise simultaneously detecting expression of any suitable internal reference gene(s). Exemplary internal reference genes include amyloid precursor protein (APP), GUSB, B2M, PSMC4, b-actin, glyceraldehyde 3-phosphate dehydrogenase (GAPDH), and ribosomal protein L37a (RPL37A). The present methods can be used for simultaneously detecting expression of 1, 2, 3, 4, 5, 6, or 7 of the above internal reference genes.

### C. Kits and Systems for Simultaneously Detecting Expression of Target Genes in a Sample

In another aspect, the present disclosure provides for a kit or system for simultaneously detecting expression of multiple target genes encoding multiple target RNA in a sample, which kit or system comprises: a) a reverse transcription primer for each of target RNA for obtaining a target cDNA corresponding to each of said target RNA via reverse transcription; b) a pair of PCR primers for amplifying each of said target cDNA to obtain an amplicon from each of said target cDNA via multiplex PCR; and d) means for analyzing said multiple amplicons using capillary electrophoresis, wherein the sizes of said multiple amplicons range from about 50 base pairs (bp) to about 300 bp, the difference of said sizes between at least two adjacent amplicons in sizes is 2 or more bp, and said 2 or more bp size difference is generated using at least one spacer nucleotide in said reverse transcription primer and/or PCR primer(s), said spacer nucleotide(s) may or may not be complementary to the nucleotide(s) at the corresponding positions(s) of the target RNA and/or target cDNA, provided that when at least one of said spacer nucleotide(s) is complementary to the nucleotide(s) at the corresponding positions(s) of the target RNA and/or target cDNA, the sizes of said multiple amplicons range from about 50 base pairs (bp) to about 150 bp.

The reverse transcription primer and the PCR primers can be designed by any suitable methods in the art. Often, factors such as amplicon length, product position, melting temperature (Tₘ) of the product, optimum annealing temperature (Tₐ Opt), and primer pair Tm mismatch calculation, can be considered in designing the primers. In some embodiments, the primers can be designed using known primer design software, e.g., Primer Premier, AlleleID®, and Beacon Designer™. Often, spacer nucleotide(s) can be added to the reverse transcription primer and/or the PCR primers to control or increase amplicon length. In some embodiments, spacer nucleotide(s) are not added to the reverse transcription primer and/or the PCR primers for any factors that are not related to amplicon length. In some embodiments, spacer nucleotide(s) are not added to the reverse transcription primer and/or the PCR primers but for the goal to control or increase amplicon length so that the lengths of various amplicons can be sufficiently distinguished in capillary electrophoresis.

The spacer nucleotide(s) can be distributed between or among the reverse transcription primer and the PCR primer(s) in any suitable manner. In some embodiments, the reverse transcription primer comprises at least one spacer nucleotide and the pair of PCR primers does not comprise any spacer nucleotide. In other embodiments, the reverse transcription primer does not comprise any spacer nucleotide and at least one of the pair of PCR primers comprises at least one spacer nucleotide. In still other embodiments, both of the pair of PCR primers comprise at least one spacer nucleotide. The spacer nucleotide(s) can be distributed between or among the PCR primers in any suitable manner. For example, the total number of the spacer nucleotides can be distributed between the two PCR primers to minimize the size difference of the two PCR primers.

In some embodiments, the reverse transcription primer comprises at least one spacer nucleotide and at least one of the pair of PCR primers comprises at least one spacer nucleotide. The spacer nucleotide(s) can be distributed between or among the reverse transcription primer and the PCR primer(s) in any suitable manner. For example, the total number of the spacer nucleotides can be distributed between the reverse transcription primer and the at least one of the pair of PCR primers to minimize the size difference of the reverse transcription primer and the at least one of the pair of PCR primers. In other embodiments, both of the pair of PCR primers comprise at least one spacer nucleotide. The spacer nucleotide(s) can be distributed between or among the PCR primers in any suitable manner. For example, the total number of the spacer nucleotides can be distributed between the two PCR primers to minimize the size difference of the two PCR primers.

The reverse transcription primer can comprise any suitable number of spacer nucleotide(s). For example, the reverse transcription primer can comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 spacer nucleotide(s). The spacer nucleotide(s) can be located at any suitable position of the reverse transcription primer. For example, the spacer nucleotide(s) can be located at the 5′ end of the reverse transcription primer.

The reverse transcription primer can comprise any suitable number of nucleotides. In some embodiments, the reverse transcription primer can comprise at least 16 nucleotides. In other embodiments, the gene specific portion of the reverse transcription primer can comprise at least 16 nucleotides. In still other embodiments, the reverse transcription primer can comprise 16 to 50 nucleotides. In yet other embodiments, the spacer nucleotide(s) is located at the 5′ end of the reverse transcription primer that comprises at least 16 nucleotides, e.g., at least 17, 18, 19, 20, 25, 30 or more nucleotides, in the non-spacer portion.

The PCR primer(s) in the pair of PCR primers can comprise any suitable number of spacer nucleotide(s). For example, one or both members of the pair of PCR primers can comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 spacer nucleotide(s). The spacer nucleotide(s) can be located at any suitable position of the PCR primer(s). For example, the spacer nucleotide(s) can be located at the 5′ end of one or both members of the pair of PCR primers.

The PCR primer(s) can comprise any suitable number of nucleotides. In some embodiments, one or both members of the pair of PCR primers can comprise at least 16 nucleotides. In other embodiments, the gene specific portion of one or both members of the pair of PCR primers can comprise at least 16 nucleotides, e.g., at least 17, 18, 19, 20, 25, 30 or more nucleotides. In still other embodiments, one or both members of the pair of PCR primers can comprise 16 to 50 nucleotides. In yet other embodiments, the spacer nucleotide(s) can be located at the 5′ end of one or both members of the pair of PCR primers that comprise at least 16 nucleotides, e.g., at least 17, 18, 19, 20, 25, 30 or more nucleotides, in the non-spacer portion.

The sizes of the multiple amplicons can have any suitable range. In some embodiments, the sizes of the multiple amplicons can range from about 50 bp to about 150 bp, e.g., about 50-140, 50-130, 50-120, 50-110 or 50-100 bp. In other embodiments, the sizes of the multiple amplicons range from about 60 bp to about 120 bp, e.g., about 60-110, 60-100, 70-120, 70-110 or 70-100 bp.

The difference of the sizes between at least two adjacent amplicons in sizes can have any suitable range. In some embodiments, the difference of the sizes between at least two adjacent amplicons in sizes can be 3 or more bp, e.g., 3, 4, 5, 6, 7, 8, 9 or 10 bp. In other embodiments, the difference of the sizes between at least a quarter of any adjacent amplicons in sizes can be 3 or more bp, e.g., 3, 4, 5, 6, 7, 8, 9 or 10 bp. In still other embodiments, the difference of the sizes between at least half of any adjacent amplicons can be 3 or more bp, e.g., 3, 4, 5, 6, 7, 8, 9 or 10 bp. In yet other embodiments, the difference of the sizes between all of any adjacent amplicons in sizes can be 3 or more bp, e.g., 3, 4, 5, 6, 7, 8, 9 or 10 bp.

In some embodiments, the reverse transcription primer is used in the reverse transcription step to obtain cDNA only, but is not used in the subsequent multiplex PCR step to obtain the amplicons. In other embodiments, the reverse transcription primer is used in the reverse transcription step to obtain cDNA, and is also used as one of the PCR primers in the subsequent multiplex PCR step to obtain the amplicons.

In some embodiments, the reverse transcription primer can further comprise a first tag sequence. In other embodiments, one or both members of the pair of PCR primers can comprise a sequence that is identical to the sequence of the first tag sequence. In still other embodiments, the other member of the pair of PCR primers can comprise a second tag sequence.

In some embodiments, one member of the pair of PCR primers comprises the reverse transcription primer that further comprises a first tag sequence and the other member of the pair of PCR primers comprises a sequence that is substantially complementary to a portion of the target cDNA and a second tag sequence. Using the above pair of PCR primers, the first PCR cycle or first two PCR cycles can generate a double-stranded DNA that comprises a sequence that is substantially complementary to the first tag sequence and the second tag sequence at its 5′ end and 3′ end, respectively. In other embodiments, the third and subsequent PCR cycles can use a pair of PCR primers that comprises one PCR primer that comprises a sequence that is identical to the sequence of the first tag sequence and another PCR primer comprises a sequence that is identical to the sequence of the second tag sequence.

In some embodiments, the present kits or systems comprise two pairs of PCR primers for amplifying each of at least a quarter, half or all of the cDNA: a) one member of the first pair of PCR primers comprising the reverse transcription primer that further comprises a first tag sequence, and the other member of the first pair of PCR primers comprising a sequence that is substantially complementary to a portion of the target cDNA and a second tag sequence; and b) one member of the second pair of PCR primers comprising a sequence that is identical to the sequence of the first tag sequence, and the other member of the second pair of PCR primers comprising a sequence that is identical to the sequence of the second tag sequence.

In some embodiments, the present kits or systems can further comprise a reverse transcription primer and a pair of PCR primers for assessing expression of an internal reference gene. The present methods can comprise reverse transcription primers and multiple pairs of PCR primers for assessing expression of at least 2, 3, 4, 5, 6, 7, 8, 9, or 10 internal reference genes.

In some embodiments, the present kits or systems can further comprise a PCR control polynucleotide for producing a control amplicon. Any suitable PCR control polynucleotide can be used, e.g., a plasmid DNA such as pcDNA.

The present kits or systems can be used for simultaneously detecting expression of any suitable number of target genes in a sample. For example, the present kits or systems can be used for simultaneously detecting expression of at least 2, 3, 4, 5, 6, 7, 8, 9, 10, 11, 12, 13, 14, 15, 16, 17, 18, 19, 20, 21, 22, 23, 24, 25, 26, 27, 28, 29 or 30 target genes in a sample.

The present kits or systems can be used for any suitable purpose. For example, the present kits or systems can be used for simultaneously detecting expression of the target genes that is associated with a therapy, e.g., a tumor or cancer therapy. In some embodiments, the present kits or systems can be used for simultaneously detecting expression of the target genes that relates to the toxicity, ADR, efficacy and/or dosage of an anti-tumor or anti-cancer drug. In other embodiments, the present kits or systems can be used for simultaneously detecting expression of the target genes that relates to the toxicity, ADR, efficacy and/or dosage of multiple anti-tumor or anti-cancer drugs.

The present kits or systems can be used for simultaneously detecting expression of any suitable target genes. Exemplary target genes include ribonucleotide reductase M1 (RRM1), topoisomerase (DNA) II alpha (TOP2A), dihydropyrimidine dehydrogenase (DPYD), excision repair cross-complementing rodent repair deficiency, complementation group 1 (ERCC1), v-erb-b2 erythroblastic leukemia viral oncogene homolog 2 (HER2), phosphatase and tensin homolog (PTEN), stathmin 1 (STMN1), thymidine phosphorylase (TYMP), kinase insert domain receptor (VEGFR), tubulin beta 3 class III (TUBB3), platelet-derived growth factor receptor, alpha polypeptide (PDGFRA), epidermal growth factor receptor (EGFR), breast cancer 1 (BRCA1), and thymidylate synthetase (TYMS). The present kits or systems can be used for simultaneously detecting expression of 2, 3, 4, 5, 6, 7, 8, 9, 10, 11, 12, 13 or 14 of the above target genes.

In some embodiments, the present kits or systems can be used for simultaneously detecting expression of any suitable internal reference gene(s). Exemplary internal reference genes include TATA-binding protein (TBP), beta-glucuronidase (GUSB), β₂ microglobulin (B2M), 26S protease regulatory subunit 6B (PSMC4), Beta-actin (b-actin), glyceraldehyde-3-phosphate dehydrogenase (GAPDH), and ribosomal protein L37a (RPL37A). The present kits or systems can be used for simultaneously detecting expression of 1, 2, 3 or 4 of the above internal reference genes.

The present kits or systems can further comprise any suitable components. For example, the present kits or systems can further comprise means for obtaining total RNA or mRNA from a sample, said total RNA or mRNA comprising target RNA encoded by multiple target genes in said sample.

The present kits or systems can be used for simultaneously detecting expression of target genes in any suitable sample. For example, the sample can be a biological sample. In some embodiments, the biological sample can be obtained or derived from a human or a non-human mammal. The present kits or systems can be used for simultaneously detecting expression of any suitable target genes in any suitable biological sample, e.g., a whole blood, a plasma, a fresh blood, a blood not containing an anti-coagulate, a urine, a saliva sample, mucosal cells, and cells from a human or a non-human mammal. In some embodiments, the sample is a formalin-fixed, paraffin-embedded (FFPE) sample.

The reverse transcription primer and/or PCR primer(s) can comprise any suitable number of spacer nucleotide(s) that is not complementary to the nucleotide(s) at the corresponding positions(s) of the target RNA and/or target cDNA. In some embodiments, at least one spacer nucleotide in the reverse transcription primer and/or PCR primer(s) is not complementary to the nucleotide(s) at the corresponding positions(s) of the target RNA and/or target cDNA. In other embodiments, at least 2, 3, 4, 5, 6, 7, 8, 9, 10, 11, 12, 13, 14, 15, 16, 17, 18, 19, 20, 21, 22, 23, 24, 25, 26, 27, 28, 29 or 30 spacer nucleotides in the reverse transcription primer and/or PCR primer(s) are not complementary to the nucleotide(s) at the corresponding positions(s) of the target RNA and/or target cDNA.

Any suitable number of the reverse transcription primers and/or PCR primers can comprise at least one spacer nucleotide that is not complementary to the nucleotide(s) at the corresponding positions(s) of the target RNA and/or target cDNA. In some embodiments, at least a quarter, half or all of the reverse transcription primers and/or PCR primers comprise at least one spacer nucleotide that is not complementary to the nucleotide(s) at the corresponding positions(s) of the target RNA and/or target cDNA. In other embodiments, at least a quarter, half or all of the reverse transcription primers and/or PCR primers comprise at least 2, 3, 4, 5, 6, 7, 8, 9, 10, 11, 12, 13, 14, 15, 16, 17, 18, 19, 20, 21, 22, 23, 24, 25, 26, 27, 28, 29 or 30 spacer nucleotides that are not complementary to the nucleotides at the corresponding positions(s) of the target RNA and/or target cDNA.

In some embodiments, the present kits can further comprise an internal reaction control RNA. Any suitable internal reaction control RNA can be used in the present kits. For example, the internal reaction control RNA can be an artificial RNA, e.g., an antisense RNA of kanamycin resistance gene.

The present kits can be used for simultaneously detecting expression of any suitable target genes. Exemplary target genes include the target genes RRM1, TOP2A, PDGFRB, ERCC1, HER2, PTEN, STMN1, ERCC2 (or XPD), VEGFR, TUBB3, DPYD, EGFR, BRCA1, and TYMS. The present methods can be used for simultaneously detecting expression of 2, 3, 4, 5, 6, 7, 8, 9, 10, 11, 12, 13 or 14 of the above target genes.

In some embodiments, the present kits can comprise simultaneously detecting expression of any suitable internal reference gene(s). Exemplary internal reference genes include APP, GUSB, B2M, PSMC4, b-actin, GAPDH, and RPL37A. The present methods can be used for simultaneously detecting expression of 1, 2, 3, 4, 5, 6, or 7 of the above internal reference genes.

### D. Polynucleotides and Primer Compositions

In yet another aspect, the present disclosure provides for an isolated polynucleotide which comprises a polynucleotide sequence that exhibits at least 70%, 75%, 80%, 85%, 90%, 95%, 99% or 100% identity to any of the RRM1, TOP2A, DPYD, ERCC1, HER2, PTEN, STMN1, TYMP, VEGFR, TUBB3, PDGFRA, EGFR, BRCA1, TYMS, TBP, GUSB, B2M and PSMC4 polynucleotide sequences set forth in Table 7, wherein said polynucleotide does not comprise a wild-type, full length RRM1, TOP2A, DPYD, ERCC1, HER2, PTEN, STMN1, TYMP, VEGFR, TUBB3, PDGFRA, EGFR, BRCA1, TYMS, TBP, GUSB, B2M and PSMC4 polynucleotide sequence from which said polynucleotide is derived.

In some embodiments, the isolated polynucleotide hybridizes to any of the RRM1, TOP2A, DPYD, ERCC1, HER2, PTEN, STMN1, TYMP, VEGFR, TUBB3, PDGFRA, EGFR, BRCA1, TYMS, TBP, GUSB, B2M and PSMC4 polynucleotide sequences set forth in Table 7 under moderately or highly stringent conditions.

In some embodiments, the isolated polynucleotide comprises any of the RRM1, TOP2A, DPYD, ERCC1, HER2, PTEN, STMN1, TYMP, VEGFR, TUBB3, PDGFRA, EGFR, BRCA1, TYMS, TBP, GUSB, B2M and PSMC4 polynucleotide sequences set forth in Table 7.

In some embodiments, the isolated polynucleotide consists essentially of any of the RRM1, TOP2A, DPYD, ERCC1, HER2, PTEN, STMN1, TYMP, VEGFR, TUBB3, PDGFRA, EGFR, BRCA1, TYMS, TBP, GUSB, B2M and PSMC4 polynucleotide sequences set forth in Table 7.

In some embodiments, the isolated polynucleotide consists of any of the RRM1, TOP2A, DPYD, ERCC1, HER2, PTEN, STMN1, TYMP, VEGFR, TUBB3, PDGFRA, EGFR, BRCA1, TYMS, TBP, GUSB, B2M and PSMC4 polynucleotide sequences set forth in Table 7.

In some embodiments, the isolated polynucleotide is complementary or substantially complementary to any of the RRM1, TOP2A, DPYD, ERCC1, HER2, PTEN, STMN1, TYMP, VEGFR, TUBB3, PDGFRA, EGFR, BRCA1, TYMS, TBP, GUSB, B2M and PSMC4 polynucleotide sequences set forth in Table 7.

In yet another aspect, the present disclosure provides for a primer composition, which primer composition comprises, consists essentially of or consists of any of the primer pairs set forth in Table 9.

In some embodiments, the primer composition comprises, consists essentially of or consists of at least 2, 3, 4, 5, 6, 7, 8, 9, 10, 11, 12, 13, 14, 15, 16, 17 and 18 of the primer pairs set forth in Table 9.

In yet another aspect, the present disclosure provides for an isolated polynucleotide which comprises a polynucleotide sequence that exhibits at least 70%, 75%, 80%, 85%, 90%, 95%, 99% or 100% identity to any of the RRM1, TOP2A, APP, PDGFRB, ERCC1, HER2, PTEN, STMN1, ERCC2, VEGFR, GUSB, TUBB3, DPYD, EGFR, B2M, BRCA1, TYMS, PSMC4, b-actin, GAPDH, RPL37A and Artificial RNA polynucleotide sequences set forth in Table 7, wherein said polynucleotide does not comprise a wild-type, full length RRM1, TOP2A, APP, PDGFRB, ERCC1, HER2, PTEN, STMN1, ERCC2, VEGFR, GUSB, TUBB3, DPYD, EGFR, B2M, BRCA1, TYMS, PSMC4, b-actin, GAPDH, RPL37A and Artificial RNA polynucleotide sequence from which said polynucleotide is derived.

In some embodiments, the polynucleotide hybridizes to any of the RRM1, TOP2A, APP, PDGFRB, ERCC1, HER2, PTEN, STMN1, ERCC2, VEGFR, GUSB, TUBB3, DPYD, EGFR, B2M, BRCA1, TYMS, PSMC4, b-actin, GAPDH, RPL37A and Artificial RNA polynucleotide sequences set forth in Table 7 under moderately or highly stringent conditions.

In some embodiments, the isolated polynucleotide comprises any of the RRM1, TOP2A, APP, PDGFRB, ERCC1, HER2, PTEN, STMN1, ERCC2, VEGFR, GUSB, TUBB3, DPYD, EGFR, B2M, BRCA1, TYMS, PSMC4, b-actin, GAPDH, RPL37A and Artificial RNA polynucleotide sequences set forth in Table 7.

In some embodiments, the isolated polynucleotide consists essentially of any of the RRM1, TOP2A, APP, PDGFRB, ERCC1, HER2, PTEN, STMN1, ERCC2, VEGFR, GUSB, TUBB3, DPYD, EGFR, B2M, BRCA1, TYMS, PSMC4, b-actin, GAPDH, RPL37A and Artificial RNA polynucleotide sequences set forth in Table 7.

In some embodiments, the isolated polynucleotide consists of any of the RRM1, TOP2A, APP, PDGFRB, ERCC1, HER2, PTEN, STMN1, ERCC2, VEGFR, GUSB, TUBB3, DPYD, EGFR, B2M, BRCA1, TYMS, PSMC4, b-actin, GAPDH, RPL37A and Artificial RNA polynucleotide sequences set forth in Table 7.

In some embodiments, the isolated polynucleotide is complementary or substantially complementary to any of the RRM1, TOP2A, APP, PDGFRB, ERCC1, HER2, PTEN, STMN1, ERCC2, VEGFR, GUSB, TUBB3, DPYD, EGFR, B2M, BRCA1, TYMS, PSMC4, b-actin, GAPDH, RPL37A and Artificial RNA polynucleotide sequences set forth in Table 7.

In yet another aspect, the present disclosure provides for a primer composition, which primer composition comprises, consists essentially of or consists of any of the primer pairs set forth in Table 7.

In some embodiments, primer composition comprises, consists essentially of or consists of at least 2, 3, 4, 5, 6, 7, 8, 9, 10, 11, 12, 13, 14, 15, 16, 17, 18, 19, 20, 21 or 22 of the primer pairs set forth in Table 7.

The polynucleotides or the primers can be made using any suitable methods. For example, the polynucleotides or the primers can be made using chemical synthesis, recombinant production or a combination thereof. See e.g., Molecular Cloning, A Laboratory Manual, Second Edition, Cold Spring Harbor Laboratory Press (1989), Current Protocols in Molecular Biology, John Wiley & Sons (1987-1997) or the like.

### E. Exemplary Embodiments

In some embodiments, the present disclosure relates to a multiplex gene expression technique, which can simultaneously or synchronously detect 30 genes of expression level with high level of specificity, sensitivity and accuracy (e.g., R²>99%), high-throughput, cost-effectiveness and time-saving, and/or low or no false-negative results.

Some of the advantages of multiplex gene expression analysis system are:

(1) High Throughput

With the capacity to analyze up to 30 genes per reaction, the scalable multiplex gene expression technique enables the examination of up to 5,760 data points unattended in 24 hours. Furthermore, this flexible system allows a user to run more than one application on the same plate and up to 192 samples unattended.

(2) High Accuracy

With unrivaled linearity (R²>0.99 for most genes), the multiplex gene expression technique delivers precise gene expression profiling that can detect gene expression changes down to 0.5 fold. A very high signal-to-noise ratio increases sensitivity and reproducibility across samples for more accurate and informative results.

(3) High Sensitivity

The multiplexing capability, coupled with capillary electrophoresis readout, can be efficiently used to look at focused sets of genes using as little as 1-5 ng of total RNA.

(4) Cost-Effectiveness

By lowering PCR expenses and improving efficiency, the multiplex power built into the multiplex gene expression technique enables a user to analyze 30 genes per sample at a dramatically reduced cost per gene expression result with considerable time savings.

(5) Comprehensive Software Tool

The multiplex gene expression technique has a sophisticated software tool that will provide an easy data management of high-throughput gene expression studies.

In some embodiments, the disclosure provides a multiplex gene expression detection method, a 14 gene expression detection kit for anticancer drugs medication guide and its detection method.

The exemplary multiplex gene expression detection method enables simultaneous detecting up to multiple RNA expression levels with high level of specificity (e.g., >99.9%), sensitivity (e.g., 1-5 ng of total RNA) and reliability, high-throughput, low cost, and low or no false-negative results. The exemplary method is based on multiplex PCR and capillary electrophoresis fragment separation. The samples analyzed could be fresh and frozen tissues, or FFPE blocks.

Exemplary, specific primers are designed to effectively amplify short nucleotide sequences in FFPE samples. While designing the primers, the amplicon size is ranging from 50 to 300 bp, e.g., 60 to 90 bp, with at least 2 bp difference between the adjacent fragments. However, to thoroughly separate the adjacent fragments on a capillary electrophoresis system, often ≧3 nt difference between adjacent fragments is required, therefore, spacers are introduced into the primer design. The spacers are served to stretch the fragment size for later CE separation, which can be 1-20 nucleotides located at the 5′-ends of the specific primers beyond 16-20 nucleotides of the primers. The spacer nucleotides can be evenly added to the 5′ end of forward and reverse primers.

The exemplary kit can synchronously detect the gene expression levels of 14 genes that are related to the toxicity/ADR, efficacy and dosage of over 20 anti-tumor drugs. The kit is often comprised of DNase/RNase Free water, 5× RT buffer, reverse transcription primers, reverse transcriptase, solution X, 10×PCR buffer, PCR primers, 25 mM magnesium chloride solution, Taq DNA Polymerase, and the positive control. The reverse transcription primers can include the reverse primers of 14 genes and 4 RNA internal reference genes. The PCR primers can include the forward primers of 14 genes, the forward primers of 4 RNA internal reference genes, and the reverse and forward primers of PCR reaction reference.

The positive control in the kit can be a mixture of RNA samples extracted from different kinds of FFPE tumor tissues.

The test process can include:

(1) Extraction of total RNA from tumor tissues including fresh and frozen tissues, or FFPE blocks.

(2) Reverse transcription using patient total RNAs as templates.

The recommended RT reaction system can be RNA sample 5-20 ng, 5×RT buffer 4 μL, RT primer mix 2 μL, reverse transcriptase 1 μL, and DEPC water to total 20 μL. The reaction condition can be 48° C. for 1 min, 42° C. for 60 min, 95° C. for 5 min and 4° C. incubation until PCR reaction started.

(3) PCR amplification.

The recommended PCR reaction system can be RT product 8.6 μL, 10×PCR buffer 2 μL, 25 mM magnesium chloride 4 μL, PCR primer mix 2 μL, Solution×2 μL, Taq DNA polymerase 1.4 μL. The reaction condition can be 95° C. for 10 min, 94° C. for 30 s, 55° C. for 30 s, 70° C. for 1 min, 35 cycles for step 2-4, 70° C. for 1 min and 4° C. incubation until capillary electrophoresis (CE) started.

(4) Signal Separation Using CE.

The recommended PCR reaction system can be PCR product 1 μL, sample loading solution (SLS) 38.75 μL, DNA marker 0.5 μL and a drop of mineral oil.

(5) Standard Curve Serial Points

The relative gene expression levels of unknown samples can be determined by calculation based on the standard curve serial points.

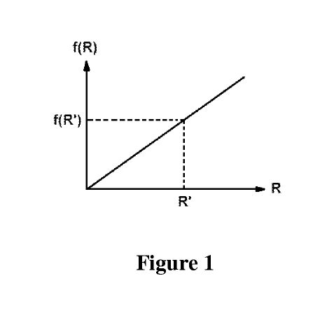

The standard curve serial points can be generated by analysis of series of 2-fold diluted FFPE RNA sample mix, containing RNA of 80 ng, 40 ng, 20 ng, 10 ng, 5 ng, 2.5 ng, 1.25 ng and 0.625 ng. Each target gene, including internal RNA reference genes, has its specific standard curve serial points (FIG. 1). We defined “R” as peak ratio of each gene and pcDNA (R=A/A_{pcDNA}), then f(R) is the function of R in the standard curve which is obtained by curve fitting of four data points which are selected in its standard curve serial points using the method like local southern according to R. Based on the standard curve of both candidate and internal reference genes, we calculated the relative expression levels “κ” of candidate genes in unknown samples as following:

κ=n*f(R_{g})/Σᵢ₌₀ⁿfᵢ(Rᵢ)(n=4)

Where R_{g} is peak ratio of a candidate gene and pcDNA, R_{g}=A_{g}/A_{pcDNA}, Rᵢ is peak ratio of an internal reference gene and pcDNA, Rᵢ=Aᵢ/A_{pcDNA}.

Compared with present technologies, the exemplary embodiment has several advantages: the exemplary embodiment enables a user to detect multiple gene expression levels (up to 30 genes) simultaneously, 1-10 internal RNA controls and reaction controls using FFPE samples as template. The relative gene expression levels of unknown samples can be determined by the calculation method based on the standard curve.

### Brief Description of the Tables

Table 1 illustrates the spacer design strategy of an exemplary, 14 gene expression detection kit.

Table 2 illustrates an exemplary, 20 μL RT reaction system of the 14 gene detection kit with pcDNA as a reaction control.

Table 3 illustrates an exemplary, RT reaction condition of incubation.

Table 4 illustrates an exemplary, 20 μL PCR reaction system of the 14 gene detection kit with pcDNA as a reaction control. pcDNA is a plasmid and added into the PCR reaction system.

Table 5 illustrates an exemplary, PCR reaction condition of the kit.

Table 6 illustrates an exemplary, PCR products CE loading.

Table 7 illustrates exemplary, primer sequences of the 14 gene expression detection kit with 7 RNA reference genes and Artificial RNA as a reaction control.

Table 8 illustrates exemplary, RT reaction system of the 14 gene detection kit with Artificial RNA as a reaction control. Artificial RNA is a RNA gene and is added into RT reaction system.

Table 9 illustrates exemplary, primer sequences of the 14 gene expression detection kit with 4 RNA reference genes and pcDNA as a reaction control.

In some embodiments, “The polymerase chain reaction (PCR)” is a biochemical technology in molecular biology to amplify a single or a few copies of a piece of DNA across several orders of magnitude, generating thousands to millions of copies of a particular DNA sequence.

In some embodiments, “capillary electrophoresis (CE)” is designed to separate species based on their size to charge ratio in the interior of a small capillary filled with an electrolyte.

In some embodiments, “Ribonucleic acid (RNA)” is a family of large biological molecules that perform multiple vital roles in the coding, decoding, regulation, and expression of genes.

In some embodiments, “Artificial RNA”, also called “internal RNA reaction control”, is an in vitro transcripted antisense RNA of a marker gene, e.g., Kanamycin resistance gene. In some embodiments, the Artificial RNA sequence is not found in nature.

In some embodiments, “Deoxyribonucleic acid (DNA)” is a molecule that encodes the genetic instructions used in the development and functioning of an organism, e.g., human beings.

In some embodiments, “primer” is a strand of nucleic acid that serves as a starting point for RNA/DNA synthesis.

In some embodiments, “cloning” refers to the fact that the method involves the replication of a single DNA molecule starting from a single living cell to generate a large population of cells containing identical DNA molecules.

In some embodiments, “multiplex technique” is a type of assay that simultaneously measures multiple analyte in a single run/cycle of the assay. It is distinguished from procedures that measure one analyte at a time.

1. Specific Primer Design

Exemplary FFPE primers are designed to effectively amplify short nucleotide sequences in FFPE samples. For this purpose, while designing FFPE primers, the amplicon size is ranging from 60 to 120 bp with 2 bp difference between the adjacent fragments. However, to thoroughly separate the adjacent fragments on a capillary electrophoresis system, ≧3 nt difference between adjacent fragments is required, therefore, spacers are introduced into the FFPE primer design. The spacers are served to stretch the fragment size for later CE separation, which are 1-20 nucleotides long located at the 5′-ends of the specific primers beyond 20 nucleotides of the primers. The spacer nucleotides are evenly added to the 5′ end of forward and reverse primers. Table 1 shows the more detailed information of primer design of the kit for FFPE samples.

TABLE 1
Spacer design strategy of the 14 gene expression detection kit

| | | gene- | Total | RT | PCR |
|---|---|---|---|---|---|
| | amplicon | specific | spacer | primer | primer |
| | with spacer | amplicon | length | length | length |
| Genes | size (bp) | size (bp) | (nt) | (nt) | (nt) |
| RRM1 | 63 | 63 | 0 | 20 | 20 |
| TBP | 66 | 65 | 1 | 21 | 20 |
| TOP2A | 69 | 67 | 2 | 21 | 21 |
| DPYD | 72 | 69 | 3 | 21 | 22 |
| ERCC1 | 75 | 71 | 4 | 22 | 22 |
| HER2 | 78 | 73 | 5 | 22 | 23 |
| PTEN | 81 | 75 | 6 | 23 | 23 |
| STMN1 | 84 | 77 | 7 | 23 | 24 |
| TYMP | 87 | 79 | 8 | 24 | 24 |
| VEGFR | 90 | 81 | 9 | 24 | 25 |
| GUSB | 93 | 83 | 10 | 25 | 25 |
| TUBB3 | 96 | 85 | 11 | 26 | 25 |
| PDGFRA | 99 | 87 | 12 | 26 | 26 |
| EGFR | 102 | 89 | 13 | 26 | 27 |
| B2M | 105 | 91 | 14 | 27 | 27 |
| BRCA1 | 108 | 93 | 15 | 27 | 28 |
| TYMS | 111 | 95 | 16 | 28 | 28 |
| PSMC4 | 114 | 97 | 17 | 28 | 29 |

2. The exemplary 14 gene expression detection kit

The present disclosure provides for an exemplary, simultaneous 14 gene expression detection kit and its detection method using FFPE sample. The kit comprises:
(1) DNase/RNase Free water;
(2) 5×RT buffer;
(3) Reverse transcription primers;
(4) Reverse transcriptase;
(5) Solution X;
(6) 10×PCR buffer;
(7) PCR primers;
(8) 25 mM magnesium chloride solution;
(9) Taq DNA polymerase;
(10) Positive control

The reverse transcription (RT) primers include the reverse primers of 14 genes and 4 RNA internal reference genes. The PCR primers include the forward primers of 14 genes, the forward primers of 4 RNA internal reference genes, and the reverse and forward primers of PCR reaction reference. The Positive Control is a mixture of RNA samples extracted from different kinds of tumor cells of FFPE samples and a plasmid pcDNA(reaction control).

The sequences of the primers are shown in Table 9 below.

3. The Exemplary Detection Processes

The exemplary detection processes are:

1. FFPE samples are collected and nucleic acids are extracted from the samples.

2. RT reaction is performed use the patient nucleic acids as the template.

(1) Make RT Reaction System (See Table 2 Below).

TABLE 2
RT reaction system

| RT Reagent | Quantity per Reaction (μL) |
|---|---|
| DNase/RNase Free Water | 8 |
| 5 × RT buffer | 4 |
| RT primer mix | 2 |
| RTase | 1 |
| RNA (5-20 ng) sample or positive control | 5 |
| Total | 20 |

(2) Mix the RT Reagents Well and Incubate as Shown in Table 3 Below.

TABLE 3
RT reaction condition

| Step | Temperature (° C.) | Time (min.) |
|---|---|---|
| 1 | 48 | 1 |
| 2 | 42 | 60 |
| 3 | 95 | 5 |
| 4 | 4 | Until PCR reaction started |

3. PCR reaction is performed use the RT product as the template.

(1) Make PCR Reaction System (See Table 4 Below).

TABLE 4
PCR reagents and sample

| PCR Reagent | Quantity per Reaction (μL) |
|---|---|
| 10 × PCR buffer | 4 |
| 25 mM MgCl₂ | 4 |
| PCR primer mix | 2 |
| Taq DNA polymerase | 1.4 |
| Solution X | 2 |
| RT product | 8.6 |
| Total | 20 |

(2) Mix the PCR Reagents Well and Incubate as Table 5 Below.

TABLE 5
PCR reaction condition

| Step | Temperature (° C.) | Time | |
|---|---|---|---|
| 1 | 95 | 10 | min |
| 2 | 94 | 30 | s |
| 3 | 55 | 30 | s |
| 4 | 70 | 1 | min |
| 5 | N/A | Repeat step 2-4 for additional 34 times | |
| 6 | 70 | 1 | min |

| | | |
|---|---|---|
| 7 | 4 | Until CE started |

4. Capillary Electrophoresis Analysis for the PCR Product.

(1) Make CE Loading Samples (See Table 6 Below).

TABLE 6
PCR products CE loading

| CE Component | Quantity per Reaction (μL) |
|---|---|
| Sample loading solution | 38.75 |
| DNA size standard 400 | 0.5 |
| PCR product | 0.1-1 |
| Mineral oil | 1 drop |

(2) CE Separation of the Sample.

5. Result Analysis

The X-axes denotes fragment size and the Y-axes denotes fragment quantity. Peak diagram is generated from the software and gene relative expression levels are calculated from the peak area data of anti-tumor drugs related genes as well as the internal reference genes. According to the standard diagram (FIG. 1), each targeted fragment should present an unsaturated and nearly equal-height peak that is narrow and single, additionally, there should be a proper interval between two neighboring peaks.

CE data normalization is done with the RNA internal controls and the internal reaction control; the relative gene expression levels of unknown samples are determined by calculation based on the standard curve.

4. Standard Curve Serial Points

The standard curve serial points are generated by analysis of series of 2-fold diluted FFPE RNA sample mix, containing RNA of 80 ng, 40 ng, 20 ng, 10 ng, 5 ng, 2.5 ng, 1.25 ng and 0.625 ng. Each target gene, including internal RNA reference genes, has its specific standard curve serial points (FIG. 1). We defined “R” as peak ratio of each gene and pcDNA (R=A/A_{pcDNA}), then f(R) is the function of R in the standard curve which is obtained by curve fitting of four data points which are selected in its standard curve serial points using the method like local southern according to R. Based on the standard curve of both candidate and internal reference genes, we calculated the relative expression levels “κ” of candidate genes in unknown samples as following:

κ=n*f(R_{g})/Σᵢ₌₀ⁿfᵢ(Rᵢ)(n=4)

Where R_{g} is peak ratio of a candidate gene and pcDNA, R_{g}=A_{g}/A_{pcDNA}, Rᵢ is peak ratio of an internal reference gene and pcDNA, Rᵢ=Aᵢ/A_{pcDNA}.

5. The Exemplary 14 Gene Expression Detection Kit with 7 Internal RNA Control Genes and Artificial RNA as Internal Reaction Control

The present exemplary embodiment is directed to a 14 gene expression detection kit with 7 internal RNA reference genes and an RNA fragment (Artificial RNA) as internal reaction control, and its detection method using FFPE sample. The kit comprises:
(1) DNase/RNase Free water;
(2) 5×RT buffer;
(3) Reverse transcription primers
(4) Artificial RNA
(5) Reverse transcriptase;
(6) Solution X;
(7) 100×PCR buffer;
(8) PCR primers;
(9) 25 mM magnesium chloride solution;
(10) Taq DNA polymerase;
(11) Positive control

The reverse transcription (RT) primers include the reverse primers of 14 genes, internal control Artificial RNA gene and 7 RNA internal reference genes. The PCR primers include the forward primers of 14 genes, the 7 RNA internal reference genes, and the reaction reference Artificial RNA gene. The Positive Control is a mixture of RNA samples extracted from different kinds of tumor cells of FFPE samples.

The sequences of the primers are shown in Table 7 below.

TABLE 7
Primer Sequences of the 14 gene expression detection kit with
7 RNA reference genes and Artificial RNA as reaction control

| Targeted | Fragment | | Count | | Count |
|---|---|---|---|---|---|
| Genes | size (nt) | RT Primers | (bp) | PCR Primers | (bp) |
| RRM1 | 103.3 | GCTACTGGCAGCTACATTGC | 20 | TCTCAGCATCGGTACAAGGC | 20 |
| | | (SEQ ID NO: 8) | | (SEQ ID NO: 30) | |
| TOP2A | 105.9 | TCGTCAGAACATGGACCCAG | 20 | TCTTCTCGGTGCCATTCAACA | 21 |
| | | (SEQ ID NO: 14) | | (SEQ ID NO: 36) | |
| APP | 109.6 | GGCCAGCATTACCATCAGTGG | 21 | GTTTGGCACTGCTCCTGCTG | 20 |
| | | (SEQ ID NO: 49) | | (SEQ ID NO: 51) | |
| PDGFRB | 112.0 | CCACTGAAGAGGGACCCATTA | 21 | AGCCAGGATCAAAGTCTCAGT | 21 |
| | | (SEQ ID NO: 50) | | (SEQ ID NO: 52) | |
| ERCC1 | 115.0 | AAGGCCTATGAGCAGAAACCAG | 22 | TTCCAGAGACCGGGAGACGAA | 21 |
| | | (SEQ ID NO: 5) | | (SEQ ID NO: 27) | |
| HER2 | 117.8 | CATTTCTGCCGGAGAGCTTTGAT | 23 | CAAACACTTGGAGCTGCTCTGG | 22 |
| | | (SEQ ID NO: 10) | | (SEQ ID NO: 32) | |
| PTEN | 120.8 | CAGATGGAAGGGGTGGAACTGTG | 23 | AACTGGCAGGTAGAAGGCAACTC | 23 |
| | | (SEQ ID NO: 4) | | (SEQ ID NO: 26) | |
| STMN1 | 122.9 | AGCTGCAGAAGAAAGACGCAAGT | 23 | AGCACTTCTTTCTCGTGCTCTCG | 23 |
| | | (SEQ ID NO: 6) | | (SEQ ID NO: 28) | |
| ERCC2 | 125.4 | AATGTCATCCAGAGCCCAGAGCA | 23 | ACGAACCAGCTGCTCACTCTGAC | 23 |
| | | (SEQ ID NO: 11) | | (SEQ ID NO: 33) | |
| VEGFR | 128.2 | GGACTTCCTGACCTTGGAGCATCT | 24 | CTGTGGATACACTTTCGCGATGCC | 24 |
| | | (SEQ ID NO: 9) | | (SEQ ID NO: 31) | |
| GUSB | 130.5 | CATGCCAGTTCCCTCCAGCTTCAAT | 25 | AGGATCACCTCCCGTTCGTACCAC | 24 |
| | | (SEQ ID NO: 15) | | (SEQ ID NO: 37) | |
| TUBB3 | 134.1 | TTGCCGCCCTCCTGCAGTATTTATG | 25 | GTCAGGCCTGGAGCTGCAATAAGAC | 25 |
| | | (SEQ ID NO: 7) | | (SEQ ID NO: 29) | |
| DPYD | 137.5 | CGGACAAGTGACCCAAGTGCTGAAG | 25 | GAAGTTCCCTACGAAGCCCTGTTTGC | 26 |
| | | (SEQ ID NO: 13) | | (SEQ ID NO: 35) | |
| EGFR | 141.5 | CCTGGACTATGTCCGGGAACACAAAGA | 27 | CGACGGTCCTCCAAGTAGTTCATGCC | 26 |
| | | (SEQ ID NO: 3) | | (SEQ ID NO: 25) | |
| B2M | 143.9 | AAAGATGAGTATGCCTGCCGTGTGAAC | 27 | GGCATCTTCAAACCTCCATGATGCTGC | 27 |
| | | (SEQ ID NO: 18) | | (SEQ ID NO: 40) | |
| BRCA1 | 146.3 | GTCCAAAGCGAGCAAGAGAATCCCAGG | 27 | CCATCCATTCCAGTTGATCTGTGGGCA | 27 |
| | | (SEQ ID NO: 1) | | (SEQ ID NO: 23) | |
| TYMS | 148.6 | AACCTAACGTGTGTTCTGGAAGGGTGTT | 28 | TTGGCATCCCAGATTTTCACTCCCTTGG | 28 |
| | | (SEQ ID NO: 12) | | (SEQ ID NO: 34) | |
| PSMC4 | 153.2 | TCAGACCAGAAGCCAGATGTGATGTACGC | 29 | CTGCTTGTAGAGCTCGAAATGCGTGAGC | 28 |
| | | (SEQ ID NO: 17) | | (SEQ ID NO: 39) | |
| b-actin | 156.5 | GGGGCGCCCCACGATGGAGGGGAAGACGG | 29 | TCACCATGGATGATGATATCGCCGCGCTC | 29 |
| | | (SEQ ID NO: 19) | | (SEQ ID NO: 43) | |
| GAPDH | 165.9 | TTACCAGAGTTAAAAGCAGCCCTGGTGAC | 29 | GGCGAGGAAGTCAGGTGGAGCGAGGCTAGC | 30 |
| | | (SEQ ID NO: 20) | | (SEQ ID NO: 44) | |
| RPL37A | 171.4 | TTCAACTCCTTCAGTCTTCTGATGGCGGAC | 30 | AGATGAAGAGACGAGCTGTGGGGATCTGGC | 30 |
| | | (SEQ ID NO: 21) | | (SEQ ID NO: 45) | |
| Artificial | 190.6 | TCATCCTGATCGACAAGACCG | 21 | CTTGCTCCTGCCGAGAAAGTA | 21 |
| RNA | | (SEQ ID NO: 22) | | (SEQ ID NO: 46) | |

The exemplary detection processes are the same to the detection processes as described above except two aspects mentioned below.

(1) Artificial RNA is used as an internal reaction control instead of pcDNA. The Artificial RNA is a RNA fragment and is added into the RT reaction (Table 8).

TABLE 8
RT reaction system of the 14 gene detection
kit with Artificial RNA as a reaction control

| | Quantity per |
|---|---|
| RT Reagent | Reaction (μL) |
| DNase/RNase Free Water | 7 |
| 5 × RT buffer | 4 |
| RT primer mix | 2 |
| Artificial RNA | 1 |
| RTase | 1 |
| RNA (5-20 ng) sample or positive control | 5 |
| Total | 20 |

(2) Artificial RNA is used to make standard curve instead of pcDNA.

The standard curve serial points are generated by analysis of series of 2-fold diluted FFPE RNA sample mix, containing RNA of 80 ng, 40 ng, 20 ng, 10 ng, 5 ng, 2.5 ng, 1.25 ng and 0.625 ng. Each target gene, including 7 internal RNA reference genes, has its specific standard curve serial points (FIG. 1). We defined “R” as peak ratio of each gene and Artificial RNA (R=A/A_{Artificial RNA}), then f(R) is the function of R in the standard curve which is obtained by curve fitting of four data points which are selected in its standard curve serial points using the method like local southern according to R. Based on the standard curve of both candidate and internal reference genes, we calculated the relative expression levels “κ” of candidate genes in unknown samples as following:

κ=n*f(R_{g})/Σᵢ₌₀ⁿfᵢ(Rᵢ)(n=4)

Where R_{g} is peak ratio of a candidate gene and Artificial RNA, R_{g}=A_{g}/A_{Artificial RNA}, Rᵢ is peak ratio of an internal reference gene and Artificial RNA, Rᵢ=Aᵢ/A_{Artificial RNA}.

The present invention is further illustrated by the following exemplary embodiments:

1. A multiplex gene expression detection technique using RNAs extracted from FFPE samples, a 14 gene expression detection kit for anticancer drugs medication guide and its detection method.

2. The multiplex gene expression detection technique of claim 1, wherein is based on multiplex PCR and capillary electrophoresis technique.

3. The multiplex gene expression detection technique of claim 1, wherein the technique enables to synchronously detect up to 30 gene expression levels including target genes, 1-10 internal RNA reference genes and a reaction control gene.

4. The multiplex gene expression detection technique of claim 1, wherein identify genes based on the fragment length showed on the graphs of capillary electrophoresis.

5. The multiplex gene expression detection technique of claim 1, wherein primers are designed to effectively amplify short nucleotide sequences in FFPE samples.

6. The multiplex gene expression detection technique of claim 1 and 5, wherein spacers are introduced into the FFPE primer design served to stretch the fragment size for later CE separation.

7. The multiplex gene expression detection technique of claim 1, 5 and 6, wherein the spacers are 1-20 nucleotides long located at the 5′-ends of the specific primers beyond 20 nucleotides of the primers.

8. The multiplex gene expression detection technique of claim 1 and 5-7, wherein the spacer nucleotides are evenly added to the 5′ end of forward and reverse primers.

9. The 14 gene expression detection kit of claim 1, wherein comprises DNase/RNase Free water, 5×RT buffer, reverse transcription primers, reverse transcriptase, solution X, 10×PCR buffer, PCR primers, 25 mM magnesium chloride solution, Taq DNA polymerase, and the positive control.

10. The 14 gene expression detection kit of claim 1, wherein the specific primers for 14 genes related to anti-cancer drug medication guide and 4 RNA internal control genes and a reaction control gene are designed.

11. The 14 gene expression detection kit of claim 1 and 10, wherein the sequences of the primers are disclosed here:

TABLE 9
Primer Sequences of the 14 gene expression detection kit
with 4 RNA reference genes and pcDNA as reaction control

| Targeted | Fragment | RT Primer | Count | PCR Primer | Count |
|---|---|---|---|---|---|
| Genes | Size (nt) | (universal primer not added) | (bp) | (universal primer not added) | (bp) |
| RRM1 | 100.5 | GCTACTGGCAGCTACATTGC | 20 | TCTCAGCATCGGTACAAGGC | 20 |
| | | (SEQ ID NO: 8) | | (SEQ ID NO: 30) | |
| TBP | 103.5 | TTTAACTTCGCTTCCGCTGG | 20 | CGCCAAGAAACAGTGATGCTG | 21 |
| | | (SEQ ID NO: 16) | | (SEQ ID NO: 38) | |
| TOP2A | 107.2 | TCGTCAGAACATGGACCCAG | 20 | TCTTCTCGGTGCCATTCAACA | 21 |
| | | (SEQ ID NO: 14) | | (SEQ ID NO: 36) | |
| DPYD | 112.4 | GCCACTGAAGAGGGACCCATTA | 22 | GAAAGCCAGGATCAAAGTCTCAGT | 24 |
| | | (SEQ ID NO: 2) | | (SEQ ID NO: 24) | |
| ERCC1 | 116.2 | AAGGCCTATGAGCAGAAACCAG | 22 | TTCCAGAGACCGGGAGACGAA | 21 |
| | | (SEQ ID NO: 5) | | (SEQ ID NO: 27) | |
| HER2 | 118.8 | CATTTCTGCCGGAGAGCTTTGAT | 23 | CAAACACTTGGAGCTGCTCTGG | 22 |
| | | (SEQ ID NO: 10) | | (SEQ ID NO: 32) | |
| PTEN | 121.6 | CAGATGGAAGGGGTGGAACTGTG | 23 | AACTGGCAGGTAGAAGGCAACTC | 23 |
| | | (SEQ ID NO: 4) | | (SEQ ID NO: 26) | |
| STMN1 | 123.8 | AGCTGCAGAAGAAAGACGCAAGT | 23 | AGCACTTCTTTCTCGTGCTCTCG | 23 |
| | | (SEQ ID NO: 6) | | (SEQ ID NO: 28) | |
| TYMP | 126.7 | AATGTCATCCAGAGCCCAGAGCA | 23 | ACGAACCAGCTGCTCACTCTGAC | 23 |
| | | (SEQ ID NO: 11) | | (SEQ ID NO: 33) | |
| VEGFR | 128.9 | GGACTTCCTGACCTTGGAGCATCT | 24 | CTGTGGATACACTTTCGCGATGCC | 24 |
| | | (SEQ ID NO: 9) | | (SEQ ID NO: 31) | |
| GUSB | 131.0 | CATGCCAGTTCCCTCCAGCTTCAAT | 25 | AGGATCACCTCCCGTTCGTACCAC | 24 |
| | | (SEQ ID NO: 15) | | (SEQ ID NO: 37) | |
| TUBB3 | 134.8 | TTGCCGCCCTCCTGCAGTATTTATG | 25 | GTCAGGCCTGGAGCTGCAATAAGAC | 25 |
| | | (SEQ ID NO: 7) | | (SEQ ID NO: 29) | |
| PDGFR | 138.8 | CGGACAAGTGACCCAAGTGCTGAAG | 25 | GAAGTTCCCTACGAAGCCCTGTTTGC | 26 |
| A | | (SEQ ID NO: 13) | | (SEQ ID NO: 35) | |
| EGFR | 142.0 | CCTGGACTATGTCCGGGAACACAAAGA | 27 | CGACGGTCCTCCAAGTAGTTCATGCC | 26 |
| | | (SEQ ID NO: 3) | | (SEQ ID NO: 25) | |
| B2M | 144.5 | AAAGATGAGTATGCCTGCCGTGTGAAC | 27 | GGCATCTTCAAACCTCCATGATGCTGC | 27 |
| | | (SEQ ID NO: 18) | | (SEQ ID NO: 40) | |
| BRCA1 | 146.7 | GTCCAAAGCGAGCAAGAGAATCCCAGG | 27 | CCATCCATTCCAGTTGATCTGTGGGCA | 27 |
| | | (SEQ ID NO: 1) | | (SEQ ID NO: 23) | |
| TYMS | 149.2 | AACCTAACGTGTGTTCTGGAAGGGTGTT | 28 | TTGGCATCCCAGATTTTCACTCCCTTGG | 28 |
| | | (SEQ ID NO: 12) | | (SEQ ID NO: 34) | |
| PSMC2 | 153.5 | TCAGACCAGAAGCCAGATGTGATGTACGC | 29 | CTGCTTGTAGAGCTCGAAATGCGTGAGC | 28 |
| | | (SEQ ID NO: 17) | | (SEQ ID NO: 39) | |
| pcDNA | 167.9 | | | TACATCAATGGGCGTGGATA | 20 |
| | | | | (SEQ ID NO: 41) | |
| | | | | GGCGGAGTTGTTACGACATT | 20 |
| | | | | (SEQ ID NO: 42) | |

12. The 14 gene expression detection kit of claim 1, wherein the positive control is a mixture of RNA samples extracted from different kinds of FFPE tumor samples and a plasmid pcDNA.

13. The 14 gene expression detection kit of claim 1, wherein the extracted RNA from both human tissue and FFPE samples can be used as a template in the kit.

### F. Example

### Example 1

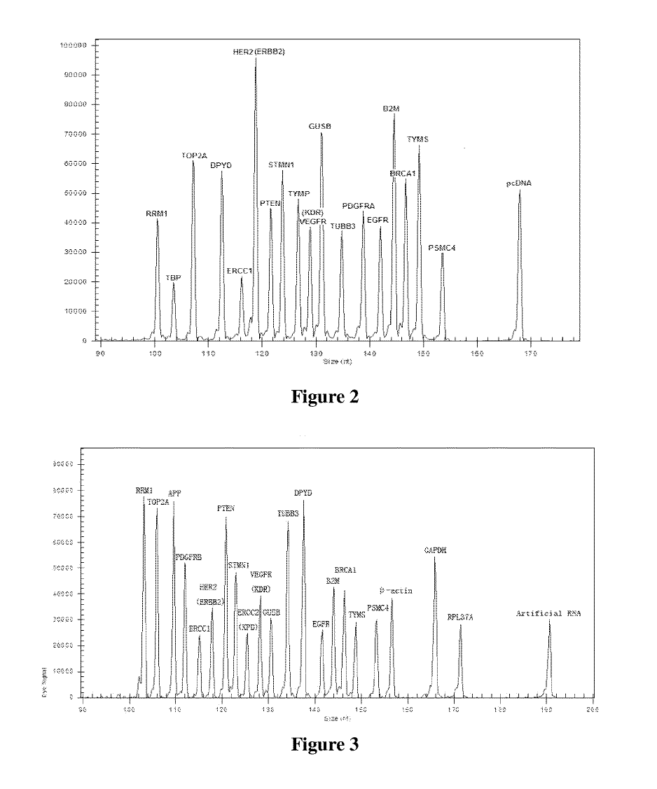

After FFPE sample collection and preparation of nucleic acids, the 14 gene expression detection kit with 4 internal RNA reference genes and pcDNA as a reaction control was used to detect the gene expression levels. RT reaction and PCR amplification (pcDNA was added into PCR mix) with patient total RNA as templates and fragment separation by capillary electrophoresis (CE) was conducted. The electrophoresis graph and analyzed results are shown in FIG. 2. Nineteen (19) peaks show up corresponding to 14 genes that are related with anticancer drug medication guide, 4 RNA reference gene peaks and a PCR control gene (pcDNA) peak. Please refer to Table 9 for the detected gene names and corresponding fragment size.

The standard curve serial points are generated by using pcDNA as control and analysis of series of 2-fold diluted FFPE RNA sample mix, containing RNA of 80 ng, 40 ng, 20 g, 10 ng, 5 ng, 2.5 ng, 1.25 ng and 0.625 ng. Each target gene, including internal RNA reference gene, got its specific standard curve serial points. Then relative RNA expression levels of genes were calculated.

### Example 2

After FFPE sample collection and preparation of nucleic acids, the 14 gene expression detection kit with 7 internal RNA reference genes and Artificial RNA as reaction control was used to detect the gene expression levels. The template was a patient total RNA and Artificial RNA was added into RT reaction. Then PCR amplification and fragment separation by capillary electrophoresis (CE) was conducted. The electrophoresis graph and analyzed results are shown in FIG. 3. Twenty-two (22) peaks corresponding to 14 genes that are related with anticancer drug medication guide, 7 RNA reference gene peaks and a reaction control gene (Artificial RNA) peak, are shown. Please refer to Table 7 for the detected gene names and corresponding fragment size.

The standard curve serial points are generated by using Artificial RNA as control and analysis of series of 2-fold diluted FFPE RNA sample mix, containing RNA of 80 ng, 40 ng, 20 g, 10 ng, 5 ng, 2.5 ng, 1.25 ng and 0.625 ng. Each target gene, including internal RNA reference gene, got its specific standard curve serial points. Then relative RNA expression levels of genes were calculated.

Citation of the above publications or documents is not intended as an admission that any of the foregoing is pertinent prior art, nor does it constitute any admission as to the contents or date of these publications or documents.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

1. A method for simultaneously detecting expression of multiple target genes in a sample, which method comprises:
a) obtaining total RNA or mRNA from a sample, said total RNA or mRNA comprising target RNA encoded by multiple target genes in said sample;
b) obtaining a target cDNA corresponding to each of said target RNA from said total RNA or mRNA via reverse transcription using said total RNA or mRNA obtained in step a) as a template and a reverse transcription primer for each of said target RNA;
c) obtaining an amplicon from each of said cDNA obtained in step b) via multiplex PCR using said cDNA as a template and a pair of PCR primers for amplifying each of said cDNA; and
d) analyzing said multiple amplicons using capillary electrophoresis,
wherein the sizes of said multiple amplicons range from about 50 base pairs (bp) to about 300 bp, the difference of said sizes between at least two adjacent amplicons in sizes is 2 or more bp, and said 2 or more bp size difference is generated using at least one spacer nucleotide in said reverse transcription primer and/or PCR primer(s), said spacer nucleotide(s) may or may not be complementary to the nucleotide(s) at the corresponding positions(s) of the target RNA and/or target cDNA, provided that when at least one of said spacer nucleotide(s) is complementary to the nucleotide(s) at the corresponding positions(s) of the target RNA and/or target cDNA, the sizes of said multiple amplicons range from about 50 base pairs (bp) to about 150 bp.

2. The method of claim 1, wherein the reverse transcription primer comprises at least one spacer nucleotide and the pair of PCR primers does not comprise any spacer nucleotide.

3. The method of claim 1, wherein the reverse transcription primer does not comprise any spacer nucleotide and at least one of the pair of PCR primers comprises at least one spacer nucleotide.

4-5. (canceled)

6. The method of claim 1, wherein the reverse transcription primer comprises at least one spacer nucleotide and at least one of the pair of PCR primers comprises at least one spacer nucleotide.

7-9. (canceled)

10. The method of claim 1, wherein the reverse transcription primer comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 spacer nucleotide(s).

11-14. (canceled)

15. The method of claim 1, wherein the spacer nucleotide(s) is located at the 5′ end of the reverse transcription primer that comprises at least 16 nucleotides in the non-spacer portion.

16. The method of claim 1, wherein the at least one of the pair of PCR primers comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 spacer nucleotide(s).

17-20. (canceled)

21. The method of claim 16, wherein the spacer nucleotide(s) is located at the 5′ end of the at least one of the pair of PCR primers that comprises at least 16 nucleotides in the non-spacer portion.

22. The method of claim 1, wherein the sizes of said multiple amplicons range from about 50 bp to about 150 bp.

23. (canceled)

24. The method of claim 1, wherein the difference of said sizes between at least two adjacent amplicons in sizes is 3 or more bp, e.g., 3, 4, 5, 6, 7, 8, 9 or 10 bp.

25-27. (canceled)

28. The method of claim 1, wherein the reverse transcription primer further comprises a first tag sequence.

29-30. (canceled)

31. The method of claim 1, wherein one member of the pair of PCR primers comprises the reverse transcription primer that further comprises a first tag sequence and the other member of the pair of PCR primers comprises a sequence that is substantially complementary to a portion of the target cDNA and a second tag sequence.

32. The method of claim 31, wherein the first PCR cycle or first two PCR cycles generate a double-stranded DNA that comprises a sequence that is substantially complementary to the first tag sequence and the second tag sequence at its 5′ end and 3′ end, respectively.

33. The method of claim 32, wherein the third and subsequent PCR cycles use a pair of PCR primers that comprises one PCR primer that comprises a sequence that is identical to the sequence of the first tag sequence and another PCR primer comprises a sequence that is identical to the sequence of the second tag sequence.

34. The method of claim 31, wherein amplification of at least a quarter, half or all of the cDNA involves the use of two pairs of PCR primers:
a) one member of the first pair of PCR primers comprising the reverse transcription primer that further comprises a first tag sequence, and the other member of the first pair of PCR primers comprising a sequence that is substantially complementary to a portion of the target cDNA and a second tag sequence; and
b) one member of the second pair of PCR primers comprising a sequence that is identical to the sequence of the first tag sequence, and the other member of the second pair of PCR primers comprising a sequence that is identical to the sequence of the second tag sequence.

35. The method of claim 1, which further comprises assessing expression of an internal reference gene.

36. (canceled)

37. The method of claim 1, which further comprises analyzing an amplicon of a PCR control polynucleotide.

38. (canceled)

39. The method of claim 1, wherein relative expression levels of multiple target genes and/or the internal reference gene(s) are assessed based on a standard curve of each of the target genes and/or the internal reference gene(s).

40. The method of claim 39, wherein the standard curve is established based on a plot between a peak ratio (“R”) of each of the target genes and/or the internal reference gene(s) over the amplicon of PCR control polynucleotide, and a function of R (f(R)).

41-42. (canceled)

43. The method of claim 1, which is used for simultaneously detecting expression of at least 2, 3, 4, 5, 6, 7, 8, 9, 10, 11, 12, 13, 14, 15, 16, 17, 18, 19, 20, 21, 22, 23, 24, 25, 26, 27, 28, 29 or 30 target genes in a sample.

44. The method of claim 43, wherein the expression of the target genes is associated with a therapy.

45. The method of claim 44, wherein the therapy is a tumor or cancer therapy.

46. The method of claim 45, wherein the expression of the target genes relates to the toxicity, ADR, efficacy and/or dosage of an anti-tumor or anti-cancer drug.

47. The method of claim 46, wherein the expression of the target genes relates to the toxicity, ADR, efficacy and/or dosage of multiple anti-tumor or anti-cancer drugs.

48. The method of claim 44, wherein the target genes are selected from the group consisting of ribonucleotide reductase M1 (RRM1), topoisomerase (DNA) II alpha (TOP2A), dihydropyrimidine dehydrogenase (DPYD), excision repair cross-complementing rodent repair deficiency, complementation group 1 (ERCC1), v-erb-b2 erythroblastic leukemia viral oncogene homolog 2 (HER2), phosphatase and tensin homolog (PTEN), stathmin 1 (STMN1), thymidine phosphorylase (TYMP), kinase insert domain receptor (VEGFR), tubulin beta 3 class III (TUBB3), platelet-derived growth factor receptor, alpha polypeptide (PDGFRA), epidermal growth factor receptor (EGFR), breast cancer 1 (BRCA1), and thymidylate synthetase (TYMS).

49. The method of claim 35, wherein the internal reference gene is selected from the group consisting of TATA-binding protein (TBP), beta-glucuronidase (GUSB), β₂ microglobulin (B2M) and 26S protease regulatory subunit 6B (PSMC4).

50. The method of claim 1, wherein the sample is a biological sample.

51. The method of claim 50, wherein the biological sample is obtained or derived from a human or a non-human mammal.

52. The method of claim 50, wherein the biological sample is selected from the group consisting of a whole blood, a plasma, a fresh blood, a blood not containing an anti-coagulate, a urine, a saliva sample, mucosal cells, and cells from a human or a non-human mammal.

53. The method of claim 1, wherein the sample is a formalin-fixed, paraffin-embedded (FFPE) sample.

54. A kit or system for simultaneously detecting expression of multiple target genes encoding multiple target RNA in a sample, which kit or system comprises:
a) a reverse transcription primer for each of target RNA for obtaining a target cDNA corresponding to each of said target RNA via reverse transcription;
b) a pair of PCR primers for amplifying each of said target cDNA to obtain an amplicon from each of said target cDNA via multiplex PCR; and
d) means for analyzing said multiple amplicons using capillary electrophoresis,
wherein the sizes of said multiple amplicons range from about 50 base pairs (bp) to about 300 bp, the difference of said sizes between at least two adjacent amplicons in sizes is 2 or more bp, and said 2 or more bp size difference is generated using at least one spacer nucleotide in said reverse transcription primer and/or PCR primer(s), said spacer nucleotide(s) may or may not be complementary to the nucleotide(s) at the corresponding positions(s) of the target RNA and/or target cDNA, provided that when at least one of said spacer nucleotide(s) is complementary to the nucleotide(s) at the corresponding positions(s) of the target RNA and/or target cDNA, the sizes of said multiple amplicons range from about 50 base pairs (bp) to about 150 bp.

55-103. (canceled)

104. The method of claim 1, wherein at least one spacer nucleotide in the reverse transcription primer and/or PCR primer(s) is not complementary to the nucleotide(s) at the corresponding positions(s) of the target RNA and/or target cDNA.

105-107. (canceled)

108. The method of claim 1, which further comprises obtaining an internal reaction control cDNA via reverse transcription using an internal reaction control RNA as a template and a reverse transcription primer for the internal reaction control RNA, obtaining an amplicon from the internal reaction control cDNA via PCR using the internal reaction control cDNA as a template and a pair of PCR primers for amplifying the internal reaction control cDNA, and analyzing the amplicon from the internal reaction control cDNA using capillary electrophoresis.

109. The method of claim 108, wherein the internal reaction control RNA is an artificial RNA, e.g., an antisense RNA of kanamycin resistance gene.

110. The method of claim 1, wherein the target genes are selected from the group consisting of RRM1, TOP2A, PDGFRB, ERCC1, HER2, PTEN, STMN1, ERCC2 (or XPD), VEGFR, TUBB3, DPYD, EGFR, BRCA1, and TYMS.

111. The method of claim 1, wherein the internal reference gene is selected from the group consisting of APP, GUSB, B2M, PSMC4, b-actin, GAPDH, and RPL37A.

112-119. (canceled)

120. An isolated polynucleotide which comprises a polynucleotide sequence that exhibits at least 70%, 75%, 80%, 85%, 90%, 95%, 99% or 100% identity to any of the RRM1, TOP2A, APP, PDGFRB, ERCC1, HER2, PTEN, STMN1, ERCC2, VEGFR, GUSB, TUBB3, DPYD, EGFR, B2M, BRCA1, TYMS, PSMC4, b-actin, GAPDH, RPL37A and Artificial RNA polynucleotide sequences set forth in Table 7, wherein said polynucleotide does not comprise a wild-type, full length RRM1, TOP2A, APP, PDGFRB, ERCC1, HER2, PTEN, STMN1, ERCC2, VEGFR, GUSB, TUBB3, DPYD, EGFR, B2M, BRCA1, TYMS, PSMC4, b-actin, GAPDH, RPL37A and Artificial RNA polynucleotide sequence from which said polynucleotide is derived.

121-125. (canceled)

126. A primer composition, which primer composition comprises, consists essentially of or consists of any of the primer pairs set forth in Table 7.

127. (canceled)
